SEQUENCE LISTING (23 sequences)

SEQ ID NO: 1 - protein, 346 aa, Homo sapiens
MGPPSAPPCRLHVPWKEVLLTASLLTFWNPPTTAKLTIESTPFNVAEGKEVLLLAHNLPQNRIGYSWYKGERVDGNSLIVGYVIGTQQATPGPAYSGRETIYPNASLLIQNVTQNDTGFYTLQVIKSDLVNEEATGQFHVYPELPKPSISSNNSNPVEDKDAVAFTCEPEVQNTTTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTMITVSGSAPVLSAVATVGITIGVLARVALI

SEQ ID NO: 2 - protein, 119 aa, synthetic construct
EVQLVESGGGLVQPGRSLKLSCAASGFTFSNYGMAWVRQAPTKGLEWVASIANSGGTTYYRDSVKGRFTFSRENAKSTLYLQMDSLRSEDTATYYCTTLKFGAGGFAYWGHGTLVTVSS

SEQ ID NO: 3 - protein, 113 aa, synthetic construct
DIVMTQTPSSQAVSAGEKVTMSCKSSQSLLYNENKKNYLAWYQQKPGQSPKLLIYWASTRESGVPDRFIGSGSGTDFTLTISSVQAEDLAVYYCQQYYIFPNTFGAGTKLELK

SEQ ID NO: 4 - protein, 117 aa, synthetic construct
QITLKESGPGILQPSQTLSLTCTFSGFSLSTYGMGVGWIRQPSGKGLEWLANIWWDDDKYYSPSLKNRLTISKDTSHNQAFLKITNVDTVDTATYYCARILLGFDYWGQGVMVTVSS

SEQ ID NO: 5 - protein, 107 aa, synthetic construct
DVQMTQSPSYLAASPGESVSISCKASKSISNTLAWYQEKPGKANKLLIHSGSALQSGTPSRFSGSGSGTDFTLTIRSLEFEDFAVYYCQHYNEYPLTFGSGTKLEIK

SEQ ID NO: 6 - protein, 8 aa, synthetic construct
GFTFSNYG

SEQ ID NO: 7 - protein, 8 aa, synthetic construct
IANSGGTT

SEQ ID NO: 8 - protein, 12 aa, synthetic construct
TTLKFGAGGFAY

SEQ ID NO: 9 - protein, 12 aa, synthetic construct
QSLLYNENKKNY

SEQ ID NO: 10 - skipped

SEQ ID NO: 11 - protein, 9 aa, synthetic construct
QQYYIFPNT

SEQ ID NO: 12 - protein, 10 aa, synthetic construct
GFSLSTYGMG

SEQ ID NO: 13 - protein, 7 aa, synthetic construct
IWWDDDK

SEQ ID NO: 14 - protein, 9 aa, synthetic construct
ARILLGFDY

SEQ ID NO: 15 - protein, 6 aa, synthetic construct
KSISNT

SEQ ID NO: 16 - skipped

SEQ ID NO: 17 - protein, 9 aa, synthetic construct
QHYNEYPLT

SEQ ID NO: 18 - protein, 10 aa, synthetic construct
  VARIANT 3: note = T or S
  VARIANT 4: note = F or L
  VARIANT 6: note = N or T
  VARIANT 9: note = M or absent
  VARIANT 10: note = G or absent
GFXXSXYGXX

SEQ ID NO: 19-22 - skipped (4 entries)

SEQ ID NO: 23 - protein, 9 aa, synthetic construct
  VARIANT 2: note = Q or H
  VARIANT 4: note = Y or N
  VARIANT 5: note = I or E
  VARIANT 6: note = F or Y
  VARIANT 8: note = N or L
QXYXXXPXT

(19) United States
(12) Patent Application Publication
(10) Pub. No.: US 2026/0347465 A1
(43) Pub. Date: Nov. 5, 2026

(54) CEACAM6 BINDING ANTIBODIES AND ANTIGEN-BINDING FRAGMENTS THEREOF

(71) Applicant: BioLegend, Inc., San Diego, CA (US)

(72) Inventors: Susannah Helene KASSMER-RODRIGUEZ, San Diego, CA (US); Weihao CHEN, San Diego, CA (US)

(21) Appl. No.: 19/485,919

(22) PCT Filed: May 21, 2024
(86) PCT No.: PCT/US2024/030383

§ 371 Date: Nov. 19, 2025

Related U.S. Application Data
(60) Provisional application No. 63/468,990, filed on May 25, 2023.

(51) Int. Cl. C07K 16/28; G01N 33/68

(52) U.S. Cl. CPC C07K 16/2803; G01N 33/6854; G01N 2333/70503

(57) ABSTRACT

Provided herein are antibodies, including antigen-binding fragments thereof, that bind all or a portion thereof of CEA-CAM6, compositions containing such antibodies or antigen-binding fragments thereof, combinations of such antibodies or antigen-binding fragments thereof and methods of use. In particular embodiments, the antibodies or antigen-binding fragments thereof are used in methods of detecting the presence of CEACAM6 through imaging, including molecular, medical and diagnostic imaging.

### RELATED PATENT APPLICATION(S)

This patent application claims the benefit of U.S. provisional patent application No. 63/468,990 filed on May 25, 2023, entitled CEACAM6 BINDING ANTIBODIES AND ANTIGEN BINDING FRAGMENTS THEREOF, naming Susannah Kassmer et al. as inventors, and designated by attorney docket no. 102738-1365417-002900US. The entire content of the foregoing patent application is incorporated herein by reference for all purposes, including all text, tables and drawings.

### FIELD

The present disclosure relates, in some aspects, to antibodies or antigen-binding fragments thereof that bind CEACAM6, as well as methods, systems and kits for detection of CEACAM6. In certain aspects, the present disclosure relates to antibodies or antigen-binding fragments thereof for use in determining levels of CEACAM6 in a sample containing or suspected of containing CEACAM6. In some aspects, the present disclosure relates to antibodies or antigen-binding fragments thereof for use in diagnosing or treating an individual with or suspected of having a disease or disorder associated with CEACAM6.

### BACKGROUND

CEACAM6, also known as CD66c, is overexpressed in several cancer types (such as, e.g., ovarian, colon, breast and non-small cell lung cancers) and promotes cancer progression by inducing epithelial-mesenchymal transition and metastasis. CEACAM6 is an immune checkpoint suppressor in hematologic malignancies.

### SUMMARY

Provided herein are antibodies, including antigen-binding fragments thereof, that bind all or a portion thereof of CEACAM6, compositions containing such antibodies or antigen-binding fragments thereof, combinations of such antibodies or antigen-binding fragments thereof and methods of use. In particular embodiments, the antibodies or antigen-binding fragments thereof are used in methods of detecting the presence of CEACAM6 through imaging, including molecular, medical and diagnostic imaging.

Provided herein are antibodies or antigen-binding fragments thereof, including those that specifically bind to a CEACAM6, such as a human CEACAM6, wherein the antibodies or antigen-binding fragments contain particular complementarity determining regions (CDRS), including heavy chain CDRS (i.e., CDRH1, CDRH2, and/or CDRH3) and light chain CDRS (i.e., CDRL1, CDRL2, and/or CDRL3), such as any described herein. in some embodiments, the antibody or antigen-binding fragment thereof includes a heavy chain variable domain and a light chain variable domain, such as any described herein.

Provided herein is an antibody or antigen-binding fragment thereof that binds CEACAM6 or a portion thereof, comprising a) an immunoglobulin heavy chain variable domain comprising (i) a heavy chain complementarity determining region 1 (CDRH1) comprising the sequence GFX1X2SX3YGX4X5 (SEQ ID NO:18), wherein X1 is T or S, X2 is F or L, X3 is N or T, X4 is M or no amino acid, and X5 is G or no amino acid; (ii) a heavy chain complementarity determining region 2 (CDRH2) comprising the sequence IX1X2X3X4X5X6X7 (SEQ ID NO:19), wherein X1 is A or W, X2 is N or W, X3 is S or D, X4 is G or D, X5 is G or D, X6 is T or K, and X7 is T or no amino acid; and (iii) a heavy chain complementarity determining region 3 (CDRH3) comprising the sequence X1X2X3X4X5GX6X7X8X9X10X11 (SEQ ID NO:20), wherein X1 is T or A, X2 is T or R, X3 is L or I, X4 is K or L, X5 is F or L, X6 is A or F, X7 is G or D, X8 is G or Y, X9 is F or no amino acid, X10 is A or no amino acid, and X11 is Y or no amino acid; and b) an immunoglobulin light chain variable domain comprising (i) a light chain complementarity determining region 1 (CDRL1) comprising the sequence X1SX2X3X4X5X6X7X8X9X10X11 (SEQ ID NO:21), wherein X1 is Q or K, X2 is L or I, X3 is L or S, X4 is Y or N, X5 is N or T, X6 is E or no amino acid, X7 is N or no amino acid, X8 is K or no amino acid, X9 is K or no amino acid, X10 is N or no amino acid, and X11 is Y or no amino acid; (ii) a light chain complementarity determining region 2 (CDRL2) comprising the sequence X1X2S (SEQ ID NO:22), wherein X1 is W or S, and X2 is A or G; and (iii) a light chain complementarity determining region 3 (CDRL3) comprising the sequence QX1YX2X3X4PX5T (SEQ ID NO:3), wherein X1 is Q or H, X2 is Y or N, X3 is I or E, X4 is F or Y, and X5 is N or L.

In some embodiments, the immunoglobulin heavy chain variable domain includes: a CDRH1 comprising the sequence of amino acids set forth in SEQ ID NO: 6 or 12, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 6 or 12; a CDRH2 comprising the sequence of amino acids set forth in SEQ ID NO: 7 or 13, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 7 or 13; and a CDRH3 comprising the sequence of amino acids set forth in SEQ ID NO: 8 or 14, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 8 or 14.

In some embodiments, the immunoglobulin heavy chain variable domain comprises: a CDRH1 comprising the sequence of amino acids set forth in SEQ ID NO: 6 or 12; a CDRH2 comprising the sequence of amino acids set forth in SEQ ID NO: 7 or 13; and a CDRH3 comprising the sequence of amino acids set forth in SEQ ID NO: 8 or 14.

In some embodiments, the immunoglobulin light chain variable domain comprises: a CDRL1 comprising the sequence of amino acids set forth in SEQ ID NO: 9 or 15, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 9 or 15; a CDRL2 comprising the sequence of amino acids set forth in SEQ ID NO: 10 or 16, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 10 or 16; and a CDRL3 comprising the sequence of amino acids set forth in SEQ ID NO: 11 or 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 11 or 17.

In some embodiments, the immunoglobulin light chain variable domain comprises: a CDRL1 comprising a sequence of amino acids set forth in SEQ ID NO: 9 or 15; a CDRL2 comprising a sequence of amino acids set forth in SEQ ID NO: 10 or 16; and a CDRL3 comprising a sequence of amino acids set forth in SEQ ID NO: 11 or 17.

In some embodiments, the CDRH1 comprises the sequence set forth in SEQ ID NO: 6, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 6, the CDRH2 comprises the sequence set forth in SEQ ID NO: 7, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 7; and the CDRH3 comprises the sequence set forth in SEQ ID NO: 8, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 8.

In some embodiments, the CDRH1 comprises the sequence set forth in SEQ ID NO: 6, the CDRH2 comprises the sequence set forth in SEQ ID NO: 7; and the CDRH3 comprises the sequence set forth in SEQ ID NO: 8.

In some embodiments, the CDRH1 comprises the sequence set forth in SEQ ID NO: 12, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 12; the CDRH2 comprises the sequence set forth in SEQ ID NO: 13 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13; and the CDRH3 comprises the sequence set forth in SEQ ID NO: 14 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14.

In some embodiments, the CDRH1 comprises the sequence set forth in SEQ ID NO: 12, the CDRH2 comprises the sequence set forth in SEQ ID NO: 13; and the CDRH3 comprises the sequence set forth in SEQ ID NO: 14.

In some embodiments, the CDRL1 comprises the sequence set forth in SEQ ID NO: 9, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 9; the CDRL2 comprises the sequence set forth in SEQ ID NO: 10 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 10; and the CDRL3 comprises the sequence set forth in SEQ ID NO: 11 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 11.

In some embodiments, the CDRL1 comprises the sequence set forth in SEQ ID NO: 9, the CDRL2 comprises the sequence set forth in SEQ ID NO: 10; and the CDRL3 comprises the sequence set forth in SEQ ID NO: 11.

In some embodiments, the CDRL1 comprises the sequence set forth in SEQ ID NO: 15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15, the CDRL2 comprises the sequence set forth in SEQ ID NO: 16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; and the CDRL3 comprises the sequence set forth in SEQ ID NO: 17 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17.

In some embodiments, the CDRL1 comprises the sequence set forth in SEQ ID NO: 15, the CDRL2 comprises the sequence set forth in SEQ ID NO: 16; and the CDRL3 comprises the sequence set forth in SEQ ID NO: 17.

In some embodiments, the CDRH1 comprises the sequence of amino acids set forth in SEQ ID NO: 6 or 12; the CDRH2 comprises the sequence of amino acids set forth in SEQ ID NO: 7 or 13; the CDRH3 comprises the sequence of amino acids set forth in SEQ ID NO: 8 or 14; the CDRL1 comprises the sequence of amino acids set forth in SEQ ID NO: 9 or 15; the CDRL2 comprises the sequence of amino acids set forth in SEQ ID NO: 10 or 16; and the CDRL3 comprises the sequence of amino acids set forth in SEQ ID NO: 11 or 17.

In some embodiments, the CDRH1 comprises the sequence set forth in SEQ ID NO: 6 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 6, the CDRH2 comprises the sequence set forth in SEQ ID NO: 7 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 7; the CDRH3 comprises the sequence set forth in SEQ ID NO: 8 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 8; the CDRL1 comprises the sequence set forth in SEQ ID NO: 9 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 9, the CDRL2 comprises the sequence set forth in SEQ ID NO: 10 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 10; and the CDRL3 comprises the sequence set forth in SEQ ID NO: 11 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 11.

In some embodiments, the CDRH1 comprises the sequence set forth in SEQ ID NO: 6, the CDRH2 comprises the sequence set forth in SEQ ID NO: 7; the CDRH3 comprises the sequence set forth in SEQ ID NO: 8; the CDRL1 comprises the sequence set forth in SEQ ID NO: 9, the CDRL2 comprises the sequence set forth in SEQ ID NO: 10; and the CDRL3 comprises the sequence set forth in SEQ ID NO: 11.

In some embodiments, the CDRH1 comprises the sequence set forth in SEQ ID NO: 12 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 12; the CDRH2 comprises the sequence set forth in SEQ ID NO: 13 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13; the CDRH3 comprises the sequence set forth in SEQ ID NO: 14 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14; the CDRL1 comprises the sequence set forth in SEQ ID NO: 15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15, the CDRL2 comprises the sequence set forth in SEQ ID NO: 16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; and the CDRL3 comprises the sequence set forth in SEQ ID NO: 17 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17.

In some embodiments, the CDRH1 comprises the sequence set forth in SEQ ID NO: 12, the CDRH2 comprises the sequence set forth in SEQ ID NO: 13; the CDRH3 comprises the sequence set forth in SEQ ID NO: 14; the CDRL1 comprises the sequence set forth in SEQ ID NO: 15, the CDRL2 comprises the sequence set forth in SEQ ID NO: 16; and the CDRL3 comprises the sequence set forth in SEQ ID NO: 17.

In some embodiments, the immunoglobulin heavy chain comprises the amino acid sequence set forth in SEQ ID NO: 2 or 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2 or 4.

In some embodiments, the immunoglobulin heavy chain comprises the amino acid sequence set forth in any of SEQ ID NO: 2 or 4.

In some embodiments, the immunoglobulin heavy chain comprises the amino acid sequence set forth in SEQ ID NO: 2, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2.

In some embodiments, the immunoglobulin heavy chain comprises the amino acid sequence set forth in any of SEQ ID NO: 2.

In some embodiments, the immunoglobulin heavy chain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 4.

In some embodiments, the immunoglobulin heavy chain comprises the amino acid sequence set forth in any of SEQ ID NO: 4.

In some embodiments, the immunoglobulin light chain comprises the amino acid sequence set forth in SEQ ID NO: 3 or 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3 or 5.

In some embodiments, the immunoglobulin light chain comprises the amino acid sequence set forth in SEQ ID NO: 3 or 5.

In some embodiments, the immunoglobulin light chain comprises the amino acid sequence set forth in SEQ ID NO: 3, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3.

In some embodiments, the immunoglobulin light chain comprises the amino acid sequence set forth in SEQ ID NO: 3.

In some embodiments, the immunoglobulin light chain comprises the amino acid sequence set forth in SEQ ID NO: 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 5.

In some embodiments, the immunoglobulin light chain comprises the amino acid sequence set forth in SEQ ID NO: 5.

In some embodiments, the immunoglobulin heavy chain comprises the amino acid sequence set forth in SEQ ID NO: 2 or 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2 or 4; and the immunoglobulin light chain comprises the amino acid sequence set forth in SEQ ID NO: 3 or 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3 or 5.

In some embodiments, the immunoglobulin heavy chain comprises the amino acid sequence set forth in SEQ ID NO: 2 or 4; and the immunoglobulin light chain comprises the amino acid sequence set forth in SEQ JD NO: 3 or 5.

In some embodiments, the immunoglobulin heavy chain comprises the amino acid sequence set forth in SEQ ID NO: 2, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2; and the immunoglobulin light chain comprises the amino acid sequence set forth in SEQ ID NO: 3, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3.

In some embodiments, the immunoglobulin heavy chain comprises the amino acid sequence set forth in SEQ ID NO: 2; and the immunoglobulin light chain comprises the amino acid sequence set forth in SEQ ID NO: 3.

In some embodiments, the immunoglobulin heavy chain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 4; and the immunoglobulin light chain comprises the amino acid sequence set forth in SEQ ID NO: 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 5.

In some embodiments, the immunoglobulin heavy chain comprises the amino acid sequence set forth in SEQ ID NO: 4; and the immunoglobulin light chain comprises the amino acid sequence set forth in SEQ ID NO: 5.

In some embodiments, the antibody or antigen-binding fragment comprises one immunoglobulin heavy chain variable domain and one immunoglobulin light chain variable domain.

In some embodiments, the antibody or antigen-binding fragment comprises two immunoglobulin heavy chain variable domains and two immunoglobulin light chain variable domains.

In some embodiments, the antibody or antigen-binding fragment thereof is isolated.

In some embodiments, the antibody or antigen-binding fragment thereof is humanized.

In some embodiments, the antibody or antigen-binding fragment thereof is conjugated.

In some embodiments, the antibody or antigen-binding fragment further comprises an oligonucleotide. In some embodiments, the oligonucleotide comprises a sample barcode sequence. In some embodiments, the oligonucleotide comprises a binding site for a primer and an anchor.

In some embodiments, the antibody or antigen-binding fragment thereof is conjugated to a detectable marker or label. In some embodiments, the detectable marker or label is conjugated directly to the antigen or antigen-binding fragment thereof. In some embodiments, the detectable marker or label is conjugated to the oligonucleotide. In some embodiments, the detectable marker or label comprises a detectable moiety. In some embodiments, the detectable moiety is a radioisotope, fluorescent label or enzyme-substrate label.

In some embodiments, the antibody or antigen-binding fragment thereof is non-diffusively immobilized on a solid support.

In some embodiments, the disclosure provides an isolated antibody that specifically binds to CEACAM6, wherein the isolated antibody competes for binding to CEACAM6 with an antibody described herein.

In some embodiments of the antibodies described herein, the antibody is a monoclonal antibody. In some embodiments, the antibody is a humanized antibody. In some embodiments, the antibody comprises one or more human framework regions. In some embodiments, the antibody or antigen-binding fragment is a single chain fragment. In some embodiments, the single chain fragment is a single chain variable fragment (scFv).

In some aspects, provided herein is a combination of antibodies or antigen-binding fragments thereof, wherein the combination comprises two or more anti-CEACAM6 antibodies or antigen-binding fragments described herein. In some embodiments, the two or more antibodies or antigen-binding fragments comprise one or more first antibody or antigen-binding fragment thereof that binds to a first epitope or region within CEACAM6; and one or more second antibody or antigen-binding fragment thereof that binds to a second epitope or region within CEACAM6. In some further embodiments, the one or more first antibody or antigen-binding fragments thereof, and the one or more second antibody or antigen-binding fragments thereof bind to a non-overlapping epitope or region of CEACAM6 (e.g., human CEACAM6) and/or do not compete for binding to CEACAM6. Further, in some embodiments, the antibody is conjugated to a detectable marker or label. In some embodiments, the at least one of the antibodies or antigen-binding fragments of the combination of two or more anti-CEACAM6 antibodies or antigen-binding fragments described herein, optionally the one or more first antibody or antigen-binding fragment thereof or the one or more second antibody or antigen-binding fragment thereof, is conjugated to a label. In some embodiments, the at least one of the antibodies or antigen-binding fragments, optionally the one or more first antibody or antigen-binding fragment thereof or the one or more second antibody or antigen-binding fragment thereof, is attached or immobilized to a solid support. In some embodiments, the one or more first or second antibody or antigen-binding fragment is attached or immobilized to a solid support and the other of the one or more first or second antibody or antigen-binding fragment is conjugated to a label. In some embodiments, the label is a fluorescent dye, a fluorescent protein, a radioisotope, a chromophore, a metal ion, gold particles, silver particles, magnetic particles, a polypeptide, an enzyme, streptavidin, biotin, a luminescent compound, or an oligonucleotide. In some embodiments, the solid support is a bead, a column, an array, an assay plate, a microwell, a stick, a filter, or a strip. In certain embodiments, the antibody is non-diffusively immobilized on a solid support. In a further embodiment, the device is a rapid detection device or a rapid diagnostic device.

In another aspect, the disclosure features an isolated nucleic acid encoding an isolated antibody described herein. The disclosure also provides an expression vector comprising the nucleic acid described herein. Further, the disclosure also provides an isolated host cell comprising the expression vector described herein.

In some embodiments, the antibody or antigen-binding fragment thereof provided herein can be used in the detection of CEACAM6 in a sample. In some embodiments, the antibody or antigen-binding fragment thereof binds to a cell expressing CEACAM6 in a sample. In some embodiments, the sample comprises immune cells. In some embodiments, the sample comprises a heterogenous population of immune cells. In some embodiments, the immune cell is selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and peripheral blood mononuclear cells (PBMCs). In some embodiments, the sample comprises a cell with a disease or disorder. In some embodiments, the disease or disorder is a cancer, an autoimmune disorder, an inflammatory disorder, a neurologic disorder, or an infection. In some embodiments, the cancer is acute myeloid leukemia, acute lymphoblastic leukemia, colorectal, ovarian, breast, gynecologic, liver, glioblastoma, Hodgkin lymphoma, chronic lymphocytic leukemia, esophagus, gastric, pancreas, colon, kidney, head and neck, lung and melanoma. In some embodiments, the detection includes the use of a single antibody or antigen-binding fragment thereof to bind a portion of CEACAM6. In some embodiments, the detection includes the use of two antibody or antigen-binding fragments thereof, each capable of binding to a different portion of CEACAM6. In some embodiments, the detection of CEACAM6 is on the surface of a cell. In some embodiments, the detection of CEACAM6 is intracellular. In some embodiments, the detection of CEACAM6 indicates the presence or absence of a disease or disorder. In some embodiments, the detection is performed in vitro. In some embodiments, the detection is performed in vivo.

In some embodiments, the antibody or antigen-binding fragment thereof binds to a CEACAM6 expressing cell.

Provided herein is a diagnostic antibody or antigen-binding fragment thereof which includes any of the antibody or antigen-binding fragment thereof described herein. Provided herein is a kit comprising the antibody or antigen-binding fragment thereof of any one of embodiments described herein. In some embodiments, the kit is a diagnostics kit configured to detect CEACAM6 in a biological sample.

Provided herein is a composition comprising the antibody or antigen-binding fragment thereof of any of the embodiments described herein and a pharmaceutically acceptable excipient. In some embodiments, the antibody or antigen-binding fragment thereof of is used as an adjuvant or in conjunction with an adjuvant.

Provided herein is an isolated nucleic acid comprising a nucleotide sequence that encodes the immunoglobulin heavy chain variable domain of the agent of any of embodiments described herein. Also provided herein is an isolated nucleic acid comprising a nucleotide sequence that encodes the immunoglobulin light chain variable domain of the agent of any of the embodiments described herein. The sequence encoding the immunoglobulin heavy chain variable domain of the agent of any of embodiments described herein and the sequence encoding the immunoglobulin light chain variable domain of the agent of any of the embodiments described herein can be on the same isolated nucleic acid or different isolated nucleic acids. Thus, also provided herein is an isolated nucleic acid comprising a nucleotide sequence that encodes the immunoglobulin heavy chain variable domain and the immunoglobulin light chain variable domain of the antibody or antigen-binding fragment thereof of any of the embodiments described herein.

Provided herein is a recombinant expression vector comprising the isolated nucleic acid of any of the embodiments described herein. Provided herein is a recombinant expression vector comprising a first expression cassette and a second expression cassette, wherein the first expression cassette includes a nucleic acid molecule comprising a nucleotide sequence that encodes the immunoglobulin heavy chain variable domain of any one of the embodiments described herein and the second expression cassette includes a nucleic acid molecule comprising a nucleotide sequence that encodes an immunoglobulin light chain variable domain of the antibody or antigen-binding fragment thereof of any one of any of the embodiments described herein.

Provided herein is a recombinant expression vector comprising a first expression cassette and a second expression cassette, wherein the first expression cassette includes a nucleic acid molecule comprising the nucleotide sequence of any of the embodiments described herein, and the second expression cassette includes a nucleic acid molecule comprising the nucleotide sequence of any of the embodiments described herein. In some embodiments, the first and second expression cassettes include a promoter.

Provided herein is a host cell transfected with the recombinant expression vector of any of the embodiments described herein.

Provided herein is an agent-drug conjugate comprising antibody or antigen-binding fragment thereof of any of the embodiments described herein. Provided herein is a composition comprising the antibody-drug conjugate and a pharmaceutically acceptable carrier,

Provided herein is a method of detecting CEACAM6 comprising a) contacting a sample with the antibody or antigen-binding fragment thereof of any of the embodiments described herein, under conditions to bind said antibody or antigen-binding fragment thereof to a CEACAM6 receptor on said sample, wherein the binding generates the production of a receptor/antibody or antigen-binding fragment thereof complex; b) detecting the presence of the receptor/antibody or antigen-binding fragment thereof complexes; and c) wherein the detecting comprises the presence or absence of the CEACAM6 receptor on said sample.

Provided herein is a method of treating or preventing a disease or disorder associated with CEACAM6 in a subject, comprising a) contacting a sample known or suspected to contain CEACAM6 with the antibody or antigen-binding fragment thereof of any of the embodiments described herein; b) detecting the presence of complexes comprising CEACAM6 and the antibody or antigen-binding fragment thereof; wherein the presence of the complexes indicates the presence of a disease or disorder; and c) administering to the subject the antibody or antigen-binding fragment thereof of any of the embodiments described herein.

Provided herein is a method of diagnosing a disease or disorder, comprising a) isolating a sample from a subject; b) incubating the sample with the antibody or antigen-binding fragment thereof of any of the embodiments described herein, for a period of time sufficient to generate CEACAM6:anti-CEACAM6 complexes; c) detecting the presence or absence of the CEACAM6:anti-CEACAM6 complexes from the isolated tissue, and d) associating presence or abundance of CEACAM6 with a location of interest of a tissue sample. In some embodiments, the increase of CEACAM6 over a control level in the location of interest of the tissue sample is indicative of a disease or disorder in a subject.

In some embodiments, the method is performed in vitro. In some embodiments, the method is performed in vivo. In some embodiments, the detection comprises intracellular detection. In some embodiments, the detection comprises detection on the surface of a cell. In some embodiments, the detection comprises hybridization of a detectable moiety to the antibody or antigen-binding fragment thereof. In some embodiments, the sample is contacted with a second antibody. In some embodiments, the second antibody is an antibody comprising a detectable moiety. In some embodiments, the detectable moiety comprises an oligonucleotide. In some embodiments, the detectable moiety comprises a fluorescent label. In some embodiments, the detecting comprises sequencing. In some embodiments, the detectable moiety comprises immunofluorescence. In some embodiments, the sample is a formalin-fixed paraffin-embedded sample. In some embodiments, the sample comprises a cell. In some embodiments, the sample comprises a tissue sample.

In some embodiments, the sample comprises immune cells. In some embodiments, the immune cell is selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, eosinophils, basophils, natural killer cells, and peripheral blood mononuclear cells (PBMCs). In some embodiments, the sample comprises a tissue or cells associated with a disease or disorder. In some embodiments, the disease or disorder is a cancer, an autoimmune disorder, an inflammatory disorder, or an infection. In some embodiments, the disease or disorder is chosen from non-viral cancers, virus-associated cancers, cancers associated with HBV infection, cancers associated with Epstein-Barr virus (EBV) infection, cancers associated with polyomavirus infection, erythema nodosum leprosum (ENL), autoimmune diseases, autoimmune inflammation, autoimmune thyroid diseases, B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, B-cell leukemias, myelodysplastic syndromes, solid phase cancer, herpes viral infections, and/or rejection of transplanted tissues or organs.

In some embodiments, the antibody or antigen-binding fragment thereof can be used in a method of associating presence or abundance of CEACAM6 with a location of interest of a tissue sample.

In some embodiments, the antibody or antigen-binding fragment thereof can be used in a method of detecting CEACAM6 in a tissue sample. In some embodiments, the method comprises generating a nucleic acid molecule comprising all or a portion of the sequence of the oligonucleotide or a complement thereof.

In some embodiments, the antibody or antigen-binding fragment thereof can be used in the construction of a protein library. In some embodiments, the construction of a protein library comprises sequencing. In some embodiments, the construction of a protein library comprises the use of flow cytometry.

The present disclosure provides isolated antibodies or antigen binding fragments that bind CEACAM6 or a portion thereof and comprise (i) an immunoglobulin heavy chain comprising a set of heavy chain complementarity determining region (CDR) amino acid sequences, CDRH1, CDRH2, and CDRH3; and, (ii) an immunoglobulin light chain comprising a set of light chain CDR amino acid sequences, CDRL1, CDRL2, and CDRL3. The sets of heavy chain and light chain CDRs are each chosen from the same of set 1 or 2:

| | CDRH1 | CDRH2 | CDRH3 | CDRL1 | CDRL2 | CDRL3 |
|---|---|---|---|---|---|---|
| CDR | (SEQ ID | (SEQ ID | (SEQ ID | (SEQ ID | (SEQ ID | (SEQ ID |
| Set | NO:) | NO:) | NO:) | NO:) | NO:) | NO:) |
| 1 | GFSLSTY | IWWDDD | ARILLGF | KSISNT | SGS (16) | QHYNEY |
| | GMG (12) | K (13) | DY (14) | (15) | | PLT (17) |
| 2 | GFTFSNY | IANSGGT | TTLKFGA | QSLLYNE | WAS (10) | QQYYIFP |
| | G (6) | T (7) | GGFAY | NKKNY | | NT (11) |
| | | | (8) | (9) | | |

In at least one embodiment, the immunoglobulin heavy chain comprises the amino acid sequence set forth in SEQ ID NO: 4, or a sequence having at least 80% amino acid sequence identity to SEQ ID NO: 4, and wherein the immunoglobulin light chain comprises the amino acid sequence set forth in SEQ ID NO: 5, or a sequence having at least 80% amino acid sequence identity to SEQ ID NO: 5.

In another embodiment, the immunoglobulin heavy chain comprises the amino acid sequence set forth in SEQ ID NO: 2, or a sequence having at least 80% amino acid sequence identity to SEQ ID NO: 2, and wherein the immunoglobulin light chain comprises the amino acid sequence set forth in SEQ ID NO: 3, or a sequence having at least 80% amino acid sequence identity to SEQ ID NO: 3.

The present disclosure features a diagnostic antibody or antigen binding fragment thereof comprising any of the embodiments described herein.

In at least one embodiment, the present disclosure provides a kit comprising the antibody or antigen binding fragment thereof of any of the embodiments described herein.

The present disclosure provides a pharmaceutical composition comprising the antibody or antigen binding fragment thereof of any of the embodiments described herein, and a pharmaceutically acceptable excipient.

Provided herein are isolated nucleic acids comprising a nucleotide sequence that encodes the immunoglobulin heavy chain variable domain of the antibodies or antigen binding fragments thereof of any of the embodiments described herein.

Provided herein is a recombinant expression vector comprising the isolated nucleic acid of any of the embodiments described herein.

The present disclosure further provides a host cell comprising the nucleic acid or the expression vector of any of the embodiments described herein.

In an embodiment, an isolated nucleic acid comprises a nucleotide sequence, wherein the nucleotide sequence encodes an immunoglobulin heavy chain comprising SEQ ID NO: 4 or a sequence having at least 90% sequence identity to the sequence set forth in SEQ ID NO: 4, or an immunoglobulin light chain comprising SEQ ID NO: 5 or a sequence having at least 90% sequence identity to the sequence set forth in SEQ ID NO: 5.

In at least one embodiment, an isolated nucleic acid comprises a nucleotide sequence, wherein the nucleotide sequence encodes an immunoglobulin heavy chain comprising SEQ ID NO: 2 or a sequence having at least 90% sequence identity to the sequence set forth in SEQ ID NO: 2, or an immunoglobulin light chain comprising SEQ ID NO: 3 or a sequence having at least 90% sequence identity to the sequence set forth in SEQ ID NO: 3.

The present disclosure features a recombinant expression vector comprising a first expression cassette and a second expression cassette, wherein the first expression cassette comprises a nucleic acid molecule comprising a nucleotide sequence that encodes the immunoglobulin heavy chain variable domain of any of the embodiments described herein, and the second expression cassette comprises a nucleic acid molecule comprising a nucleotide sequence that encodes an immunoglobulin light chain variable domain of the antibody or antigen binding fragment thereof of any of the embodiments described herein.

Provided herein is a method of detecting CEACAM6, where the method includes: contacting a sample with the antibody or antigen binding fragment thereof of any of the embodiments described herein, under conditions to bind said antibody or antigen binding fragment thereof to a CEACAM6 receptor in said sample, wherein the binding generates the production of a receptor/antibody or antigen binding fragment thereof complex.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1: Anti-CEACAM6 antibodies AB1 and AB2 did not show any binding to CEACAM8 (CD66b) on RBL-1 cells transfected with CEACAM8. The histograms of the negative controls and of the anti-CEACAM6 antibodies show a comparable single peak in the low fluorescence intensity range indicating the absence of any bound anti-CEACAM6 antibody. RBL-1 cells were transfected with CEACAM8 and stained with two different amounts of anti-CEACAM6 antibody AB1, AB2 and commercially available anti-CEACAM6 antibody REA414 and (iv) anti-CEACAM1/5/6 antibody ASL-32, whereby the commercially available antibodies served as negative controls. As isotype control Allophycocyanin (APC)-labeled anti-human IgG1 recombinant antibody (BioLegend cat no 403506, clone QA16A12) was used. Anti-CEACAM6 antibodies were detected using an APC-labeled anti-rat IgG secondary antibody (APC-hCD66c, X-axis).

FIG. 2: No staining of lymphocytes by AB1 and AB 2 was observed based on the very low number of fluorescent signals in the top right quadrants Q2 and Q6, respectively. FIG. 2A: lymphocytes were co-stained with anti-human CEACAM6 antibody AB1, which was detected using a PE-labeled anti-rat IgG secondary antibody (PE-A, X-axis in left and right density plot), and either an anti-CD19 antibody labeled with Brilliant Violet 605™ (CD19 BV605, Y-axis, left density plot) or an anti-CD3 antibody labeled with fluorescein isothiocyanate (CD3 FITC, Y-axis, right density plot). FIG. 2B: lymphocytes were co-stained with anti-human CEACAM6 antibody clone AB2, which was detected using a PE-labeled anti-rat IgG secondary antibody (PE-A, X-axis in left and right density plot), and either CD19 BV605 (left density plot) or CD3 FITC (right density plot).

FIG. 3: Both anti-CEACAM6 antibodies AB1 and AB2 detected CEACAM6 on granulocytes and monocytes based on the high fluorescent signal density in the top right Q2 quadrants. Peripheral blood leukocytes were stained with anti-human CEACAM6 antibody AB1 (FIG. 3A) and anti-human CEACAM6 antibody clone AB2 (FIG. 3B), which were detected using a PE-labeled anti-rat IgG secondary antibody (PE-A, X-axis in left and right density plots). For detection of anti-CEACAM6 antibodies bound to CEACAM6 on monoctyes, cells were co-stained with APC-Cy7-labeled anti-CD14 antibody (APC-Cy7, Y-axis, left density plot). For detection of anti-CEACAM6 antibodies bound to CEACAM6 on granulocytes, cells were gated for side scatter (SSC, Y-axis, right density plot).

FIG. 4: Color dot plots from a titration experiment show that at 0.1 ug both anti-CEACAM6 antibodies AB1 and AB2 exhibited a higher fluorescence intensity than both reference clones ASL-32 and REA414 (see black squared boxes). Peripheral blood leukocytes were stained with seven different amounts of anti-human CEACAM6 antibody AB1 (bottom row), AB2 (second last row), reference clone REA414 (second row) or reference clone ASL-32 (top row) ranging from 0.001 ug to 1 ug. Anti-CEACAM6 antibodies were detected using an APC-labeled anti-rat IgG secondary antibody (APC-hCD66c, Y-axis).

FIG. 5: Both anti-CEACAM6 antibodies AB1 (FIG. 5B) and AB2 (FIG. 5A) blocked the binding of the PE-labeled reference clone KOR-SA3544 (KOR-SA3544-PE) to CEACAM6 on granulocytes as indicated by the left shift of the light grey peak. Peripheral blood leukocytes were either directly stained with PE-labeled reference clone KOR-SA3544 (KOR-SA3544-PE, Comp-PE-A, X-axis, dark grey histogram) or first pre-incubated with anti-human CEACAM6 antibody AB1 (FIG. 5B) or AB2 (FIG. 5A) followed by staining with KOR-SA3544-PE (light grey histogram). Data shown is gated on granulocyte population.

FIG. 6: Both anti-CEACAM6 antibodies AB1 and AB2 blocked binding of the CEACAM1/5/6-specific reference antibody ASL-32 to CEACAM6 as indicated by the left shift of the light grey peak in FIG. 6C and FIG. 6D. AB1 and AB2 did not block binding of the CEACAM8 (CD66b)-specific reference antibody 6/40c-PE to CD66b as indicated by the light grey and dark grey peak lying on top of each other in FIG. 6A and FIG. 6B. Peripheral blood leukocytes were incubated with AB1 or AB2 for 15 minutes followed by staining with PE-labeled reference antibody ASL-32 (ASL-32-PE) or PE-labeled reference antibody 6/40c (6/40c-PE). Data shown is gated on granulocyte population.

FIG. 7: Titration curve shows that both anti-CEACAM6 antibodies AB1 and AB2 are at least two-fold brighter than the reference antibody KOR-SA3544 at a concentration of antibody per million cells of 1. Peripheral blood leukocytes from two different donors were stained with PE-labeled AB1 (AB1, light grey graph) or PE-labeled AB2 (AB2, dark grey graph) or PE-labeled KOR-SA3544 (KOR-SA3544, grey triangular data point). Data shown is gated on granulocyte population.

FIG. 8: Anti-CEACAM6 antibody AB1 reacted with CEACAM6 on A549 cells as indicated by the right shift of the AB1-PE peak compared to the Isotype control peak in FIG. 8A. AB1 had no cross-reactivity with other CEACAM family members as indicated by the AB1-PE peak and the Isotype control peak lying on top of each other in FIGS. 8B-E. Anti-CEACAM6 antibody AB2 reacted with both CEACAM6 and CEACAM4 as indicated by the right shift of the AB2-PE peak compared to the Isotype control peak in FIGS. 8A, 8B and 8D. Cell lines A549, U937, A431, THP-1 and LNCaP were stained with PE-labeled AB1 (AB1-PE) or PE-labeled AB2 (AB2-PE) or PE-labeled rat IgG2a, k isotype control (Isotype control).

FIG. 9: Anti-CEACAM6 antibody AB1 detected expression of CEACAM6 in colon epithelial cells by immunohistochemistry (IHC) as indicated by the fluorescent signals in the highlighted areas (left fluorescence image, white squares). AB2 was not able to detect expression of CEACAM6 as indicated by the lack of any fluorescent signals (right fluorescence image). Human paraffin embedded colon tissue was subjected to heat-mediated antigen retrieval with Sodium Citrate and incubated with 5 μg/ml purified anti-human CEACAM6 antibody AB1 (FIG. 9 left) and AB2 (FIG. 9 right) followed by staining using an anti-rat IgG Alexa Fluor®555 secondary antibody. Nuclei were counterstained with DAPI dye. The image was captured by a 10× objective.

FIG. 10: Anti-CEACAM6 antibody AB1 detected the expression of CEACAM6 in human lung adenocarcinoma cell line A549 by immunocytochemistry (ICC) as indicated by the fluorescent signals in the left and middle fluorescence image. A549 cells were grown on 96-well plates with coverslip bottom and were fixed with Fixation Buffer (cat no 420801) for 30 minutes. Cells were washed twice with PBS and stained with 5 ug/ml Ultra-LEAF™ purified anti-human CEACAM6 antibody AB1 followed by staining with Alexa Fluor®555-labeled anti-rat IgG secondary antibody (cat #405420). Rat IgG2a, k isotype control antibody was used as negative control (Isotype, FIG. 10 right fluorescence image). Nuclei were counterstained with DAPI dye (cat no 422801). Cells were imaged using a 40×objective.

FIG. 11: Anti-CEACAM6 antibody AB1 blocked invasion of lung adenocarcinoma cells as indicated by a significant reduction of number of cells/field for all three AB1 concentrations tested. The effect of different concentrations of Ultra-LEAF™ purified anti-human CEACAM6 antibody AB1 on the ability of inhibiting cells to invade through the extracellular matrix was tested by a Matrigel invasion assay. As CEACAM6 antibody reference the commercially available antibody 1H7-4B (Reference) was used at 20 μg/ml. The human lung adenocarcinoma cell line A549 was serum starved for 24 h and seeded onto the top well of Corning® BioCoat Matrigel® Invasion Chamber. Rat IgG2a, k isotype antibody was used as control (Isotype). DMEM media with 20% serum was placed in the bottom well as chemoattractant. After 16 h, cells that had migrated to the bottom of the membrane were counted.

FIG. 12: Anti-CEACAM6 antibody AB1 blocked migration of lung adenocarcinoma cells. The effect of Ultra-LEAF™ purified anti-human CEACAM6 antibody AB1 on the ability of blocking cells to migrate was tested by a wound healing assay. The human lung adenocarcinoma cell line A549 was grown to confluence, and a line was scratched using a 10 ul pipette tip. Rat IgG2a, k isotype antibody was used as control (Isotype). After 16 h, the width of the gap was measured using Image J, and % gap closure was calculated normalized to isotype controls.

FIG. 13: Cross-linking of CEACAM6 with AB1 induced signaling by activating Akt phosphorylation and increased actin polymerization as indicated by the significantly increased fluorescence signals and intensity when comparing the bottom right versus left and top right versus left fluorescence images. The effect of AB1 to induce CEACAM6 signaling by cross-linking was tested on the human lung adenocarcinoma cell line A549. Cells were grown on 96-well plates with coverslip bottom and treated with 5 μg/ml Ultra-LEAF™ purified anti-human CEACAM6 antibody AB1 or rat IgG2a, k isotype control (Isotype) for 15 minutes at 37 °C followed by anti-rat IgG secondary antibody (5 ug/ml) for 30 minutes at 37 °C. Cells were fixed with Fixation Buffer for 30 minutes. Cells were washed twice with 1× Intracellular Staining Permeabilization Wash Buffer and then stained with anti-AKT Phospho (Ser473) antibody (antibody A21001C) followed by Alexa Fluor®555 anti-mouse IgG antibody, Flash Phalloidin™ Red 594 dye to image filamentous F-actin and DAPI dye to image nuclei.

### DETAILED DESCRIPTION

CEACAM6 is a part of the carcinoembryonic antigen (CEA) family and is a glycosyl phosphatidyl inositol (GPI) anchored cell surface glycoprotein. CEACAM6 has 84% amino acid overlap with CEACAM1, 84-66% overlap with CEACAM5, 79% overlap with CEAMCAM8, 68% overlap with CEACAM7, 67% overlap with CEACAM2 and 48% overlap with CEACAM4. CEACAM6 is a cell adhesion molecule that mediates homotypic binding with other CEA family members and heterotypic binding with integrin receptors. The biological role of CEACAM6 includes development of neural tissue, inflammation, immune cell transmigration and immune response. CEACAM6 overexpressed in more than 50% of all human adenocarcinomas. CEACAM6 promotes aberrant cell differentiation, anti-apoptosis, cell growth and resistance to therapeutic agents, cell invasion and metastasis. As described herein, in in vitro studies antibodies directed against CEACAM6 inhibited cell migration, invasion and adhesion. Further, BAY1834942 is a novel checkpoint inhibitor with potential for the treatment of patients with CEACAM6 expressing cancers (trial completed in 2021, no results posted). CEACAM6 is expressed on the epithelial surfaces and is myeloid restricted in bone marrow and blood and expressed in granulocytes and monocytes. CEACAM6 is a myeloid marker which has aberrant expression in pre-B cell ALL, with strong correlation with non-random genetic changes (BCR/ABL rearrangement).

Provided herein are antibodies that bind CEACAM6, including antigen-binding fragments thereof, nucleic acids encoding such antibodies and antigen-binding fragments, and cells, such as recombinant cells for expressing and production of these antibodies and antigen-binding fragments that can bind to CEACAM6 under physiological and/or in vitro conditions. Also provided are methods of producing and using the antibodies and antigen-binding fragments such as in methods for detecting CEACAM6 in a sample from an individual, including methods for laboratory/research purposes (e.g., flow cytometry, ELISA, and/or Western blot), and/or for the use and treatment and/or prevention of various diseases or disorders through the delivery of pharmaceutical or other compositions that contain such antibodies or antigen-binding fragments thereof.

All references cited herein, including patent applications, patent publications, and scientific literature and databases, are herein incorporated by reference in their entirety for all purposes to the same extent as if each individual reference were specifically and individually indicated to be incorporated by reference.

For clarity of disclosure, and not by way of limitation, the detailed description is divided into the subsections that follow. The section headings used herein are for organizational purposes only and are not to be construed as limiting the subject matter described.

### I. DEFINITIONS

Unless defined otherwise, all terms of art, notations and other technical and scientific terminology used herein are intended to have the same meaning as is commonly understood by one of ordinary skill in the art to which the subject matter pertains. In some cases, terms with commonly understood meanings are defined herein for clarity and/or for ready reference, and the inclusion of such definitions herein should not necessarily be construed to represent a substantial difference over what is generally understood in the art. It is to be understood that the disclosure provided herein is not limited to particular compositions or biological systems. It is also understood that the terminology used herein is for the purpose of describing particular embodiments only, and is not to be construed as limiting.

The term “antibody” as used herein includes antigen-binding fragments thereof that retain binding specificity. For example, there are a number of well characterized antigen-binding fragments. Thus, for example, pepsin digests an antibody C-terminal to the disulfide linkages in the hinge region to produce F(ab)′2, a dimer of Fab which itself is a light chain joined to VH—CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially an Fab with part of the hinge region (see, Fundamental Immunology, W. E. Paul, ed., Raven Press, N.Y. (1993), for a more detailed description of other antigen-binding fragments). While various antigen-binding fragments are defined in terms of the digestion of an intact antibody, one of skill will appreciate that fragments can be synthesized de novo either chemically or by utilizing recombinant DNA methodology. Thus, the term antibody, as used herein also includes antigen-binding fragments either produced by the modification of whole antibodies or synthesized using recombinant DNA methodologies.

An antibody as described herein can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, the antibody is IgG (e.g., IgG1, IgG2, IgG3, IgG4), IgM, IgA, IgD, or IgE.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

In an antibody, substitution variants have at least one amino acid residue removed and a different residue inserted in its place. The sites of greatest interest for substitutional mutagenesis include the hypervariable regions, but framework alterations are also contemplated. Examples of conservative substitutions are described herein.

Substantial modifications in the biological properties of the antibody are accomplished by selecting substitutions that differ significantly in their effect on maintaining (a) the structure of the polypeptide backbone in the area of the substitution, for example, as a 3-sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Len, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.
   Non-conservative substitutions are made by exchanging a member of one of these classes for another class.

One type of substitution that can be made is to change one or more cysteines in the antibody, which may be chemically reactive, to another residue, such as, without limitation, alanine or serine. For example, there can be a substitution of a non-canonical cysteine. The substitution can be made in a CDR or framework region of a variable domain or in the constant region of an antibody. In some embodiments, the cysteine is canonical (e.g., involved in di-sulfide bond formation). Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antigen-binding fragment such as an Fv fragment.

Antibodies include V_{H}-V_{L} dimers, including single chain antibodies (antibodies that exist as a single polypeptide chain), such as single chain Fv antibodies (sFv or scFv) in which a variable heavy and a variable light chain domains are joined together (directly or through a peptide linker) to form a continuous polypeptide. The single chain Fv antibody is a covalently linked V_{H}-V_{L} which may be expressed from a nucleic acid including V_{H}- and V_{L}-encoding sequences either joined directly or joined by a peptide-encoding linker (e.g., Huston, et al. Proc. Nat. Acad. Sci. USA, 85:5879-5883, 1988). While the V_{H} and V_{L} are connected to each as a single polypeptide chain, the V_{H} and V_{L} domains associate non-covalently. Alternatively, the antibody can be another fragment. Other fragments can also be generated, e.g., using recombinant techniques, as soluble proteins or as fragments obtained from display methods. Antibodies can also include diantibodies and miniantibodies. Antibodies of the disclosure also include heavy chain dimers, such as antibodies from camelids. In some embodiments an antibody is dimeric. In other embodiments, the antibody may be in a monomeric form that has an active isotype. In some embodiments the antibody is in a multivalent form, e.g., a trivalent or tetravalent form.

An “antibody fragment” or “antigen-binding fragment thereof” comprises a portion of an intact antibody, the antigen-binding and/or the variable region of the intact antibody. Antibody fragments or antigen-binding fragments thereof, include but are not limited to Fab fragments, Fab′ fragments, F(ab′)₂ fragments, Fv fragments, disulfide-linked Fvs (dsFv), Fd fragments, Fd′ fragments; diabodies; linear antibodies (see U.S. Pat. No. 5,641,870, Example 2; Zapata et al, Protein Eng. 8(10): 1057-1062 [1995]); single-chain antibody molecules, including single-chain Fvs (scFv) or single-chain Fabs (scFab); antigen-binding fragments of any of the above and multispecific antibodies from antibody fragments.

“Fv” is composed of one heavy- and one light-chain variable region domain linked by non-covalent association. From the folding of these two domains emanate six complementarity determining regions (CDR) (3 in each from the heavy and light chain) that contribute the amino acid residues for antigen binding and confer antigen-binding specificity to the antibody. However, even a single variable domain (or half of an Fv comprising only three CDRs specific for an antigen) has the ability to recognize and bind antigen, although, in some cases, at a lower affinity than the entire binding site.

“dsFv” refers to an Fv with an engineered intermolecular disulfide bond, which stabilizes the VH-VL pair.

An “Fd fragment” is a fragment of an antibody containing a variable domain (VH) and one constant region domain (CHI) of an antibody heavy chain.

A “Fab fragment” is an antibody fragment that results from digestion of a full-length immunoglobulin with papain, or a fragment having the same structure that is produced synthetically, e.g., by recombinant methods. A Fab fragment contains a light chain (containing a VL and CL) and another chain containing a variable domain of a heavy chain (V_{H}) and one constant region domain of the heavy chain (CHI).

A “F(ab′)2 fragment” is an antibody fragment that results from digestion of an immunoglobulin with pepsin at pH 4.0-4.5, or a fragment having the same structure that is produced synthetically, e.g., by recombinant methods. The F(ab′)2 fragment essentially contains two Fab fragments where each heavy chain portion contains an additional few amino acids, including cysteine residues that form disulfide linkages joining the two fragments.

A “Fab′ fragment” is a fragment containing one half (one heavy chain and one light chain) of the F(ab′)2 fragment.

An “Fd′ fragment” is a fragment of an antibody containing one heavy chain portion of a F(ab′)2 fragment.

An “Fv′ fragment” is a fragment containing only the VH and VL domains of an antibody molecule.

An “scFv fragment” refers to an antibody fragment that contains a variable light chain (VL) and variable heavy chain (VH), covalently connected by a polypeptide linker in any order. The linker is of a length such that the two variable domains are bridged without substantial interference. Exemplary linkers are (Gly-Ser)n residues with some Glu or Lys residues dispersed throughout to increase solubility.

“Diabodies” are dimeric scFv; diabodies typically have shorter peptide linkers than scFvs, and preferentially dimerize.

As used herein, the terms “variable region” and “variable domain” refer to the portions of the light and heavy chains of an antibody that include amino acid sequences of complementarity determining regions (CDRs, e.g., HCDR1, HCDR2, HCR3, LCDR1, LCDR2, and LCDR3) and framework regions (FRs). The variable domain for the heavy and light chains is commonly designated V_{H} and V_{L}, respectively. The variable domain is included on Fab, F(ab′)₂, Fv and scFv antigen-binding fragments described herein, and is involved in specific antigen recognition.

As used herein, “complementarity-determining region (CDR)” refers to the three hypervariable regions in each chain that interrupt the four framework regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three-dimensional space.

The amino acid sequences of the CDRs and framework regions can be determined using various well-known definitions in the art, e.g., Kabat, North method (see, e.g., North et al., J Mol Biol. 406(2):228-256, 2011), Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

As used herein, “chimeric antibody” refers to an immunoglobulin molecule in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region, or portion thereof, having a different or altered antigen specificity; or with corresponding sequences from another species or from another antibody class or subclass.

As used herein, “humanized antibody” refers to an immunoglobulin molecule in CDRs from a donor antibody are grafted onto human framework sequences. Humanized antibodies may also comprise residues of donor origin in the framework sequences. The humanized antibody can also comprise at least a portion of a human immunoglobulin constant region. Humanized antibodies may also comprise residues which are found neither in the recipient antibody nor in the imported CDR or framework sequences. Humanization can be performed using methods known in the art (e.g., Jones et al., Nature 321:522-525; 1986; Riechmann et al., Nature 332:323-327, 1988; Verhoeyen et al., Science 239:1534-1536, 1988); Presta, Curr. Op. Struct. Biol. 2:593-596, 1992; U.S. Pat. No. 4,816,567), including techniques such as “superhumanizing” antibodies (Tan et al., J. Immunol. 169: 1119, 2002) and “resurfacing” (e.g., Staelens et al., Mol. Immunol. 43: 1243, 2006; and Roguska et al., Proc. Natl. Acad. Sci USA 91: 969, 1994).

The term “recombinant” when used with a reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “antigen,” “immunogen,” “antibody target,” “target analyte,” and like terms are used herein to refer to a molecule, compound, or complex that is recognized by an antibody, i.e., can be specifically bound by the antibody. The term can refer to any molecule that can be specifically recognized by an antibody, e.g., a polypeptide, polynucleotide, carbohydrate, lipid, chemical moiety, or combinations thereof (e.g., phosphorylated or glycosylated polypeptides, etc.). One of skill will understand that the term does not indicate that the molecule is immunogenic in every context, but simply indicates that it can be targeted by an antibody.

Antibodies bind to an “epitope” on an antigen. The epitope is the localized site on the antigen that is recognized and bound by the antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

A “label” or a “detectable moiety” is a diagnostic agent or component detectable by spectroscopic, radiological, photochemical, biochemical, immunochemical, chemical, or other physical means. Exemplary labels include radiolabels (e.g., ¹¹¹In, ^{99m}Tc, ¹³¹I, ⁶⁷Ga) and other FDA-approved imaging agents. Additional labels include ³²P, fluorescent dyes, electron-dense reagents, enzymes, biotin, digoxigenin, or haptens and proteins or other entities which can be made detectable, e.g., by incorporating a radiolabel into the targeting agent. Any method known in the art for conjugating a nucleic acid or nanocarrier to the label may be employed, e.g., using methods described in Hermanson, Bioconjugate Techniques 1996, Academic Press, Inc., San Diego.

A “labeled” or “tagged” antibody or agent is one that is bound, either covalently, through a linker or a chemical bond, or noncovalently, through ionic, van der Waals, electrostatic, or hydrogen bonds to a label such that the presence of the antibody or agent may be detected by detecting the presence of the label bound to the antibody or agent.

Techniques for conjugating detectable and therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The terms “specific for,” “specifically binds,” and like terms refer to a molecule (e.g., antibody or antigen-binding fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. For example, an antibody that specifically binds a target (e.g., TMPRSS2) will typically bind the target with at least a 2-fold greater affinity than a non-target. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

The term “binds” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least 2/3 of the antibody targets in a solution (e.g., at least any of 75, 80, 85, 90, 91, 92, 93, 94, 95, 96, 97, 98, 99, or 100%). One of skill will recognize that some variability will arise depending on the method and/or threshold of determining binding.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value or a range gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” As described herein, the preferred algorithms can account for gaps and the like. Preferably, identity exists over a region that is at least about 25 amino acids or nucleotides in length, or more preferably over a region that is 50-100 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, is a reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art.

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the disclosure. Software for performing BLAST analyses is publicly available through the National Center for Biotechnology Information (//www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation I of 10, M=5, N=−4 and a comparison of both strands. For amino acid sequences, the BLASTP program uses as defaults a wordlength of 3, and expectation I of 10, and the BLOSUM62 scoring matrix (see Henikoff & Henikoff, Proc. Natl. Acad. Sci. USA 89:10915 (1989)) alignments (B) of 50, expectation I of 10, M=5, N=−4, and a comparison of both strands.

The term “nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

The terms “polypeptide,” “peptide” and “protein” are used interchangeably herein to refer to a polymer of amino acid residues. The terms encompass to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “compete,” as used herein with regard to an antibody, means that a first antibody, or an antigen-binding portion thereof, competes for binding with a second antibody, or an antigen-binding portion thereof, where binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present disclosure. Regardless of the mechanism by which such competition or cross-competition occurs (e.g., steric hindrance, conformational change, or binding to a common epitope, or portion thereof, and the like), the skilled artisan would appreciate, based upon the teachings provided herein, that such competing and/or cross-competing antibodies are encompassed and can be useful for the methods disclosed herein.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253 (1983)); solid phase direct biotin-avidin EIA (see Kirkland et al., J. Immunol. 137:3614-3619 (1986)); solid phase direct labeled assay, solid phase direct labeled sandwich assay (see Harlow and Lane, Antibodies, A Laboratory Manual, Cold Spring Harbor Press (1988)); solid phase direct label RIA using I-125 label (see Morel et al., Molec. Immunol. 25(1):7-15 (1988)); solid phase direct biotin-avidin EIA (Cheung et al., Virology 176:546-552 (1990)); and direct labeled RIA (Moldenhauer et al., Scand. J. Immunol. 32:77-82 (1990)). Typically, such an assay involves the use of purified antigen bound to a solid surface or cells bearing either of these, an un-labeled test immunoglobulin and a labeled reference immunoglobulin. Competitive inhibition is measured by determining the amount of label bound to the solid surface or cells in the presence of the test immunoglobulin. Usually the test immunoglobulin is present in excess. Antibodies identified by competition assay (competing antibodies) include antibodies binding to the same epitope as the reference antibody and antibodies binding to an adjacent epitope sufficiently proximal to the epitope bound by the reference antibody for steric hindrance to occur. Usually, when a competing antibody is present in excess, it will inhibit specific binding of a reference antibody to a common antigen by at least 50 or 75%.

The term “CEACAM6” as used herein refers to human CEACAM6 proteins, isoforms or variants thereof, including naturally occurring variants of human CEACAM6, such as splice variants or allelic variants. The amino acid sequence of an exemplary human CEACAM6 is shown in SEQ ID NO: 1. In some embodiments, human CEACAM6 can refer to a variant, such as an allelic variant or splice variant, that exhibits at least or at least about 85%, 86%, 87%, 88%, 89%, 90%, 91%, 92%, 93%, 94%, 95%, 96%, 97%, 98%, or 99% sequence identity to any of SEQ ID NO: 1. In some embodiments, it is understood that the provided antibodies or antigen-binding fragments may exhibit cross-reactive binding to another mammalian CEACAM6 protein, such as murine CEACAM6, or a primate CEACAM6.

Human CEACAM6 Uniprot P40199
(SEQ ID NO: 1)
MGPPSAPPCR LHVPWKEVLL TASLLTFWNP PTTAKLTIES TPFNVAEGKE
VLLLAHNLPQ NRIGYSWYKG ERVDGNSLIV GYVIGTQQAT PGPAYSGRET
IYPNASLLIQ NVTQNDTGFY TLQVIKSDLV NEEATGQFHV YPELPKPSIS
SNNSNPVEDK DAVAFTCEPE VQNTTTTYLWWV NGQSLPVSPR LQLSNGNMTL
TLLSVKRNDA GSYECEIQNP ASANRSDPVT LNVLYGPDVP TISPSKANYR
PGENLNLSCH AASNPPAQYS WFINGTFQQS TQELFIPNIT VNNSGSYMCQ
AHNSATGLNR TTVTMITVSG SAPVLSAVAT VGITIGVLAR VALI

By “solid support” is meant a non-aqueous matrix to which an antibody according to the provided disclosure can adhere or attach. For example, solid supports include, but are not limited to, a microtiter plate, a membrane (e.g., nitrocellulose), a bead, a dipstick, a thin-layer chromatographic plate, or other solid medium.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

### II. ANTIBODIES THAT BIND CEACAM6

Provided herein are antibodies, including antigen-binding fragments thereof, that specifically bind to CEACAM6. In some embodiments, the provided antibodies include monoclonal antibodies and antigen-binding fragments thereof that bind CEACAM6 and provide superior target specificity, signal-to-noise ratios, and the like as compared to other reported antibodies. Also provided herein are methods for producing anti-CEACAM6 antibodies, and methods for detecting and using such antibodies.

The carcinoembryonic antigen-related cell adhesion molecules (CEACAMs) are a large family of proteins in humans. The CEACAM family consists of membrane-linked glycoproteins anchored to the cell surface either by glycophosphatidyl-inositol (GPI) anchor or a transmembrane domain. Carcinoembryonic antigen-related cell adhesion molecule 6 (CEACAM6) is a member of the family normally expressed on epithelial and myeloid cell surfaces and is linked to the cell surface by a GPI anchor (Johnson and Mahadevan, Clinical Cancer Drugs 2, pp. 100-11 (2015)). CEACAM6 is associated with invasion and metastasis in several cancers, such as pancreatic, lung, and colon cancer cells (Wu, et al., Translational Oncology 14, 101057 (2021)). CEACAM6 modulates cancer progression through aberrant cell differentiation, anti-apoptosis, cell growth and resistance to therapeutic agents (Johnson and Mahadevan, Clinical Cancer Drugs 2, pp. 100-11 (2015)).

CEACAM6 antibodies with no cross-reactivity towards other members of the CEACAM6 protein family, and with the ability to block CEACAM6 function and inhibit cancer cell invasion are provided in the present disclosure.

In some embodiments, any of the antibodies or antigen-binding fragments thereof provided herein bind all or a portion of CEACAM6. In some embodiments, any of the antibodies of antigen-binding fragments thereof provided herein bind all or a portion of the extracellular domain of CEACAM6.

In some embodiments, any of the antibodies or antigen-binding fragments thereof is a CEACAM6 antibody or antigen-binding fragment thereof. In some embodiments, the antibody or antigen-binding fragment thereof is isolated (e.g., separated from a component of its natural environment (e.g., an animal, a biological sample)). In some embodiments, the anti-antibody is a humanized antibody, or an antigen-binding fragment thereof. In some embodiments, the antibody is a derivative of a humanized antibody that binds. In some embodiments, the antibody binds under laboratory conditions (e.g., binds in vitro, binds in a flow cytometry assay, binds in an ELISA). In some embodiments, the antibody binds under physiological conditions (e.g., binds in a cell in a subject).

Generally, the antibodies provided herein comprise at least one immunoglobulin heavy chain variable domain and at least one immunoglobulin light chain variable domain. In some embodiments, an antibody described herein comprises two immunoglobulin heavy chain variable domains and two immunoglobulin light chain variable domains. Typically, each immunoglobulin heavy chain variable domain of the antibody comprises first, second, and third heavy chain complementarity determining regions (CDRs; CDRH1, CDRH2, and CDRH3), and each immunoglobulin light chain variable domain of the antibody comprises first, second, and third light chain CDRs (CDRL1, CDRL2, and CDRL3).

In some embodiments, the antibodies are antigen-binding fragments such as Fab, F(ab′)2, Fv or scFv. The antigen-binding fragments can be generated using any means known in the art including, chemical digestion (e.g., papain or pepsin) and recombinant methods. Methods for isolating and preparing recombinant nucleic acids are known to those skilled in the art (see, Sambrook et al., Molecular Cloning. A Laboratory Manual (2d ed. 1989); Ausubel et al., Current Protocols in Molecular Biology (1995)). The antibodies can be expressed in a variety of host cells, including E. coli, other bacterial hosts, yeast, and various higher eukaryotic cells such as the COS, CHO, and HeLa cells lines and myeloma cell lines.

The disclosure provides an isolated antibody or antigen-binding fragment thereof that specifically binds CEACAM6 or a portion thereof, comprising a) an immunoglobulin heavy chain variable domain comprising: (i) a heavy chain complementarity determining region 1 (CDRH1) comprising the sequence GFX1X2SX3YGX4X5 (SEQ ID NO:18), wherein X1 is T or S, X2 is F or L, X3 is N or T, X4 is M or no amino acid, and X5 is G or no amino acid; (ii) a heavy chain complementarity determining region 2 (CDRH2) comprising the sequence IX1X2X3X4X5X6X7 (SEQ ID NO:19), wherein X1 is A or W, X2 is N or W, X3 is S or D, X4 is G or D, X5 is G or D, X6 Is T or K, and X7 is T or no amino acid; and (iii) a heavy chain complementarity determining region 3 (CDRH3) comprising the sequence X1X2X3X4X5GX6X7X8X9X10X11 (SEQ ID NO:20), wherein X1 is T or A, X2 is T or R, X3 is L or I, X4 is K or L, X5 is F or L, X6 is A or F, X7 is G or D, X8 is G or Y, X9 is F or no amino acid, X10 is A or no amino acid, and X11 is Y or no amino acid; and b) an immunoglobulin light chain variable domain comprising: (i) a light chain complementarity determining region 1 (CDRL1) comprising the sequence X1SX2X3X4X5X6X7X8X9X10X11 (SEQ ID NO:21), wherein X1 is Q or K, X2 is L or I, X3 is L or S, X4 is Y or N, X5 is N or T, X6 is E or no amino acid, X7 is N or no amino acid, X8 is K or no amino acid, X9 is K or no amino acid, X10 is N or no amino acid, and X11 is Y or no amino acid; (ii) a light chain complementarity determining region 2 (CDRL2) comprising the sequence X1X2S (SEQ ID NO:22), wherein X1 is W or S, and X2 is A or G; and (iii) a light chain complementarity determining region 3 (CDRL3) comprising the sequence QX11YX2X3X4PX5T (SEQ ID NO:3), wherein X1 is Q or H, X2 is Y or N, X3 is I or E, X4 is F or Y, and X5 is N or L.

In some embodiments, antibodies of the disclosure can comprise sequences of a heavy chain complementarity determining region 1 (CDRH1), an CDRH2, an CDRH3, a light chain complementarity determining region 1 (CDRL1), a CDRL2, a CDRL3. Exemplary CDR amino acid sequences and associated SEQ ID NOs and exemplary amino acid sequences for heavy chain variable domains (VH), and light chain variable domains (VL) and associated SEQ ID NOs are set forth in Table 1.

TABLE 1
Amino acid sequences of CEACAM6, heavy chain and light chain variable
domains and CDRs from anti-CEACAM6 antibodies.

| SEQ ID | | |
|---|---|---|
| NO. | Sequence | Description |
| 1 | MGPPSAPPCR LHVPWKEVLL TASLLTFWNP | Human |
| | PTTAKLTIES TPFNVAEGKE VLLLAHNLPQ | CEACAM6 |
| | NRIGYSWYKG ERVDGNSLIV GYVIGTQQAT | Uniprot |
| | PGPAYSGRET IYPNASLLIQ NVTQNDTGFY | P40199 |
| | TLQVIKSDLV NEEATGQFHV YPELPKPSIS | |
| | SNNSNPVEDK DAVAFTCEPE VQNTTTTYLWWV | |
| | NGQSLPVSPR LQLSNGNMTL TLLSVKRNDA | |
| | GSYECEIQNP ASANRSDPVT LNVLYGPDVP | |
| | TISPSKANYR PGENLNLSCH AASNPPAQYS | |
| | WFINGTFQQS TQELFIPNIT VNNSGSYMCQ | |
| | AHNSATGLNR TTVTMITVSG SAPVLSAVAT | |
| | VGITIGVLAR VALI | |
| 2 | EVQLVESGGGLVQPGRSLKLSCAAS GFTFSNYG | Anti-Human |
| | MAWVRQAPTKGLEWVAS IANSGGTT | CEACAM6 |
| | YYRDSVKGRFTFSRENAKSTLYLQMDSLRSEDTATYYC | Heavy Chain |
| | TTLKFGAGGFAY WGHGTLVTVSS | AB2 |
| 3 | DIVMTQTPSSQAVSAGEKVTMSCKSS | Anti-Human |
| | QSLLYNENKKNY LAWYQQKPGQSPKLLIY WAS | CEACAM6 |
| | TRESGVPDRFIGSGSGTDFTLTISSVQAEDLAVYYC | Light Chain |
| | QQYYIFPNT FGAGTKLELK | AB2 |
| 4 | QITLKESGPGILQPSQTLSLTCTFS GFSLSTYGMG | Anti-Human |
| | VGWIRQPSGKGLEWLAN IWWDDDK | CEACAM6 |
| | YYSPSLKNRLTISKDTSHNQAFLKITNVDTVDTATYYC | Heavy Chain |
| | ARILLGEDY WGQGVMVTVSS | AB1 |
| 5 | DVQMTQSPSYLAASPGESVSISCKAS KSISNT | Anti-Human |
| | LAWYQEKPGKANKLLIH SGS | CEACAM6 |
| | ALQSGTPSRFSGSGSGTDFTLTIRSLEFEDFAVYYC | Light Chain |
| | QHYNEYPLT FGSGTKLEIK | AB1 |
| 6 | GFTFSNYG | CDRH1 |
| | | AB2 Anti- |
| | | Human |
| | | CEACAM6 |
| | | Antibody |
| 7 | IANSGGTT | CDRH2 |
| | | AB2 Anti- |
| | | Human |
| | | CEACAM6 |
| | | Antibody |
| 8 | TTLKFGAGGFAY | CDRH3 |
| | | AB2 Anti- |
| | | Human |
| | | CEACAM6 |
| | | Antibody |
| 9 | QSLLYNENKKNY | CDRL1 |
| | | AB2 Anti- |
| | | Human |
| | | CEACAM6 |
| | | Antibody |
| 10 | WAS | CDRL2 |
| | | AB2 Anti- |
| | | Human |
| | | CEACAM6 |
| | | Antibody |
| 11 | QQYYIFPNT | CDRL3 |
| | | AB2 Anti- |
| | | Human |
| | | CEACAM6 |
| | | Antibody |
| 12 | GFSLSTYGMG | CDRH1 |
| | | AB1 Anti- |
| | | Human |
| | | CEACAM6 |
| | | Antibody |
| 13 | IWWDDDK | CDRH2 |
| | | AB1 Anti- |
| | | Human |
| | | CEACAM6 |
| | | Antibody |
| 14 | ARILLGFDY | CDRH3 |
| | | AB1 Anti- |
| | | Human |
| | | CEACAM6 |
| | | Antibody |
| 15 | KSISNT | CDRL1 |
| | | AB1 Anti- |
| | | Human |
| | | CEACAM6 |
| | | Antibody |
| 16 | SGS | CDRL2 |
| | | AB1 Anti- |
| | | Human |
| | | CEACAM6 |
| | | Antibody |
| 17 | QHYNEYPLT | CDRL3 |
| | | AB1 Anti- |
| | | Human |
| | | CEACAM6 |
| | | Antibody |
| 18 | GFX1X2SX3YGX4X5 | CDRH1 |
| | X1 is T or S, X2 is F or L, X3 is N or T, X4 is M or | |
| | no amino acid, and X5 is G or no amino acid | |
| 19 | IX1X2X3X4X5X6X7 | CDRH2 |
| | X1 is A or W, X2 is N or W, X3 is S or D, X4 is G or | |
| | D, X5 is G or D, X6 is T or K, and X7 is T or no amino | |
| | acid | |
| 20 | X1X2X3X4X5GX6X7X8X9X10X11 | CDRH3 |
| | X1 is T or A, X2 is T or R, X3 is L or I, X4 is K or | |
| | L, X5 is F or L, X6 is A or F, X7 is G or D, X8 is G | |
| | or Y, X9 is F or no amino acid, X10 is A or no amino | |
| | acid, and X11 is Y or no amino acid | |
| 21 | X1SX2X3X4X5X6X7X8X9X10X11 | CDRL1 |
| | X1 is Q or K, X2 is L or I, X3 is L or S, X4 is Y or N, | |
| | X5 is N or T, X6 is E or no amino acid, X7 is N or no | |
| | amino acid, X8 is K or no amino acid, X9 is K or no | |
| | amino acid, X10 is N or no amino acid, and X11 is Y or | |
| | no amino acid | |
| 22 | X1X2S | CDRL2 |
| | X1 is W or S, and X2 is A or G | |
| 23 | QX1YX2X3X4PX5T | CDRL3 |
| | X1 is Q or H, X2 is Y or N, X3 is I or E, X4 is F or | |
| | Y, X5 is N or L | |

In some embodiments, the antibody or antigen-binding fragments thereof comprises: (i) an immunoglobulin heavy chain variable domain comprising a set of heavy chain complementarity determining region (CDR) amino acid sequences, CDRH1, CDRH2, and CDRH3; and, (ii) an immunoglobulin light chain variable domain comprising a set of light chain CDR amino acid sequences, CDRL1, CDRL2, and CDRL3, wherein the sets of heavy chain and light chain CDRs are each chosen from the same of set 1 or 2 set forth in Table 2.

TABLE 2

| | CDRH1 | CDRH2 | CDRH3 | CDRL1 | CDRL2 | CDRL3 |
|---|---|---|---|---|---|---|
| CDR | (SEQ ID | (SEQ ID | (SEQ ID | (SEQ ID | (SEQ ID | (SEQ ID |
| Set | NO:) | NO:) | NO:) | NO:) | NO:) | NO:) |
| 1 | GFSLSTY | IWWDDD | ARILLGF | KSISNT | SGS (16) | QHYNEY |
| | GMG (12) | K (13) | DY (14) | (15) | | PLT (17) |
| 2 | GFTFSNY | IANSGGT | TTLKFGA | QSLLYNE | WAS (10) | QQYYIFP |
| | G (6) | T (7) | GGFAY | NKKNY | | NT (11) |
| | | | (8) | (9) | | |

In some embodiments, the immunoglobulin heavy chain variable domain includes: a CDRH1 comprising the sequence of amino acids set forth in SEQ ID NO: 6 or 12, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 6 or 12; a CDRH2 comprising the sequence of amino acids set forth in SEQ ID NO: 7 or 13, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 7 or 13; and a CDRH3 comprising the sequence of amino acids set forth in SEQ ID NO: 8 or 14, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 8 or 14.

In some embodiments, the immunoglobulin heavy chain variable domain comprises: a CDRH1 comprising the sequence of amino acids set forth in SEQ ID NO: 6 or 12; a CDRH2 comprising the sequence of amino acids set forth in SEQ ID NO: 7 or 13; and a CDRH3 comprising the sequence of amino acids set forth in SEQ ID NO: 8 or 14.

In some embodiments, the immunoglobulin light chain variable domain comprises: a CDRL1 comprising the sequence of amino acids set forth in SEQ ID NO: 9 or 15, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 9 or 15; a CDRL2 comprising the sequence of amino acids set forth in SEQ ID NO: 10 or 16, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 10 or 16; and a CDRL3 comprising the sequence of amino acids set forth in SEQ ID NO: 11 or 17, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 11 or 17.

In some embodiments, the immunoglobulin light chain variable domain comprises: a CDRL1 comprising a sequence of amino acids set forth in SEQ ID NO: 9 or 15; a CDRL2 comprising a sequence of amino acids set forth in SEQ ID NO: 10 or 16; and a CDRL3 comprising a sequence of amino acids set forth in SEQ ID NO: 11 or 17.

In some embodiments, the CDRH1 comprises the sequence set forth in SEQ ID NO: 6, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 6, the CDRH2 comprises the sequence set forth in SEQ ID NO: 7, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 7; and the CDRH3 comprises the sequence set forth in SEQ ID NO: 8, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 8.

In some embodiments, the CDRH1 comprises the sequence set forth in SEQ ID NO: 6, the CDRH2 comprises the sequence set forth in SEQ ID NO: 7; and the CDRH3 comprises the sequence set forth in SEQ ID NO: 8.

In some embodiments, the CDRH1 comprises the sequence set forth in SEQ ID NO: 12, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 12; the CDRH2 comprises the sequence set forth in SEQ ID NO: 13 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13; and the CDRH3 comprises the sequence set forth in SEQ ID NO: 14 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14.

In some embodiments, the CDRH1 comprises the sequence set forth in SEQ ID NO: 12, the CDRH2 comprises the sequence set forth in SEQ ID NO: 13; and the CDRH3 comprises the sequence set forth in SEQ ID NO: 14.

In some embodiments, the CDRL1 comprises the sequence set forth in SEQ ID NO: 9, or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 9; the CDRL2 comprises the sequence set forth in SEQ ID NO: 10 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 10; and the CDRL3 comprises the sequence set forth in SEQ ID NO: 11 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 11.

In some embodiments, the CDRL1 comprises the sequence set forth in SEQ ID NO: 9, the CDRL2 comprises the sequence set forth in SEQ ID NO: 10; and the CDRL3 comprises the sequence set forth in SEQ ID NO: 11.

In some embodiments, the CDRL1 comprises the sequence set forth in SEQ ID NO: 15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15, the CDRL2 comprises the sequence set forth in SEQ ID NO: 16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; and the CDRL3 comprises the sequence set forth in SEQ ID NO: 17 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17.

In some embodiments, the CDRL1 comprises the sequence set forth in SEQ ID NO: 15, the CDRL2 comprises the sequence set forth in SEQ ID NO: 16; and the CDRL3 comprises the sequence set forth in SEQ ID NO: 17.

In some embodiments, the CDRH1 comprises the sequence of amino acids set forth in SEQ ID NO: 6 or 12; the CDRH2 comprises the sequence of amino acids set forth in SEQ ID NO: 7 or 13; the CDRH3 comprises the sequence of amino acids set forth in SEQ ID NO: 8 or 14; the CDRL1 comprises the sequence of amino acids set forth in SEQ ID NO: 9 or 15; the CDRL2 comprises the sequence of amino acids set forth in SEQ ID NO: 10 or 16; and the CDRL3 comprises the sequence of amino acids set forth in SEQ ID NO: 11 or 17.

In some embodiments, the immunoglobulin heavy chain comprises the amino acid sequence set forth in SEQ ID NO: 2 or 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2 or 4.

In some embodiments, the immunoglobulin heavy chain comprises the amino acid sequence set forth in any of SEQ ID NO: 2 or 4.

In some embodiments, the immunoglobulin light chain comprises the amino acid sequence set forth in SEQ ID NO: 3 or 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3 or 5.

In some embodiments, the immunoglobulin light chain comprises the amino acid sequence set forth in SEQ ID NO: 3 or 5.

In some embodiments, the immunoglobulin heavy chain comprises the amino acid sequence set forth in SEQ ID NO: 2 or 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2 or 4; and the immunoglobulin light chain comprises the amino acid sequence set forth in SEQ ID NO: 3 or 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3 or 5.

In some embodiments, the immunoglobulin heavy chain comprises the amino acid sequence set forth in SEQ ID NO: 2 or 4; and the immunoglobulin light chain comprises the amino acid sequence set forth in SEQ ID NO: 3 or 5.

### AB2

In some embodiments, the CDRH1 comprises the sequence set forth in SEQ ID NO: 6 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 6, the CDRH2 comprises the sequence set forth in SEQ ID NO: 7 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 7; the CDRH3 comprises the sequence set forth in SEQ ID NO: 8 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 8; the CDRL1 comprises the sequence set forth in SEQ ID NO: 9 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 9, the CDRL2 comprises the sequence set forth in SEQ ID NO: 10 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 10; and the CDRL3 comprises the sequence set forth in SEQ ID NO: 11 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 11.

In some embodiments, the CDRH1 comprises the sequence set forth in SEQ ID NO: 6, the CDRH2 comprises the sequence set forth in SEQ ID NO: 7; the CDRH3 comprises the sequence set forth in SEQ ID NO: 8; the CDRL1 comprises the sequence set forth in SEQ ID NO: 9, the CDRL2 comprises the sequence set forth in SEQ ID NO: 10; and the CDRL3 comprises the sequence set forth in SEQ ID NO: 11.

In some embodiments, the immunoglobulin heavy chain comprises the amino acid sequence set forth in SEQ ID NO: 2, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2.

In some embodiments, the immunoglobulin heavy chain comprises the amino acid sequence set forth in any of SEQ ID NO: 2.

In some embodiments, the immunoglobulin light chain comprises the amino acid sequence set forth in SEQ ID NO: 3, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3.

In some embodiments, the immunoglobulin light chain comprises the amino acid sequence set forth in SEQ ID NO: 3.

In some embodiments, the immunoglobulin heavy chain comprises the amino acid sequence set forth in SEQ ID NO: 2, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 2; and the immunoglobulin light chain comprises the amino acid sequence set forth in SEQ ID NO: 3, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 3.

In some embodiments, the immunoglobulin heavy chain comprises the amino acid sequence set forth in SEQ ID NO: 2; and the immunoglobulin light chain comprises the amino acid sequence set forth in SEQ ID NO: 3.

### AB1

In some embodiments, the CDRH1 comprises the sequence set forth in SEQ ID NO: 12 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 12; the CDRH2 comprises the sequence set forth in SEQ ID NO: 13 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 13; the CDRH3 comprises the sequence set forth in SEQ ID NO: 14 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 14; the CDRL1 comprises the sequence set forth in SEQ ID NO: 15 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 15, the CDRL2 comprises the sequence set forth in SEQ ID NO: 16 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 16; and the CDRL3 comprises the sequence set forth in SEQ ID NO: 17 or a sequence of amino acids that exhibits at least 80%, 81%, 82%, 83%, 84%, 85%, 86%, 87%, 88%, 89%, 90%, 91%, 92%, 93%, 94%, 95%, 96%, 97%, 98%, 99% or more sequence identity to SEQ ID NO: 17.

In some embodiments, the CDRH1 comprises the sequence set forth in SEQ ID NO: 12, the CDRH2 comprises the sequence set forth in SEQ ID NO: 13; the CDRH3 comprises the sequence set forth in SEQ ID NO: 14; the CDRL1 comprises the sequence set forth in SEQ ID NO: 15, the CDRL2 comprises the sequence set forth in SEQ ID NO: 16; and the CDRL3 comprises the sequence set forth in SEQ ID NO: 17.

In some embodiments, the immunoglobulin heavy chain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 4.

In some embodiments, the immunoglobulin heavy chain comprises the amino acid sequence set forth in any of SEQ ID NO: 4.

In some embodiments, the immunoglobulin light chain comprises the amino acid sequence set forth in SEQ ID NO: 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 5.

In some embodiments, the immunoglobulin light chain comprises the amino acid sequence set forth in SEQ ID NO: 5.

In some embodiments, the immunoglobulin heavy chain comprises the amino acid sequence set forth in SEQ ID NO: 4, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 4; and the immunoglobulin light chain comprises the amino acid sequence set forth in SEQ ID NO: 5, or a sequence having at least 80%, 81%, 82%, 83%, 84%, 85%, 86%, 87%, 88%, 89%, 90%, 91%, 92%, 93%, 94%, 95%, 96%, 97%, 98%, 99% or 100% amino acid sequence identity to SEQ ID NO: 5.

In some embodiments, the immunoglobulin heavy chain comprises the amino acid sequence set forth in SEQ ID NO: 4; and the immunoglobulin light chain comprises the amino acid sequence set forth in SEQ ID NO: 5.

### Fc Polypeptide

An antibody provided herein can comprise a fragment crystallizable region (Fc region), also referred to as an Fe polypeptide herein. An Fe polypeptide is part of each of the two heavy chains in the antibody and can interact with certain cell surface receptors and certain components of the complement system. An Fc polypeptide typically includes the CH2 domain and the CH3 domain, which are immunoglobulin constant region domain polypeptides. In some embodiments, the Fc polypeptide in an antibody described herein can be a wild-type Fe polypeptide, e.g., a human IgG1 Fe polypeptide.

In other embodiments, an antibody described herein can comprise a variant of the wild-type Fc polypeptide that has at least 90% (e.g., 90%, 91%, 92%, 93%, 94%, 95%, 96%, 97%, 98%, 99%, or 99.5%) identity to the sequence of a wild-type Fe polypeptide (e.g., SEQ ID NO:87) and at least one amino acid substitution relative to the sequence of a wild-type Fc polypeptide.

In some embodiments, an Fe polypeptide includes one or more modifications (e.g., one or more amino acid substitutions, insertions, or deletions relative to a comparable wild-type Fe region). Antibodies comprising modified Fc polypeptides typically have altered phenotypes relative to antibodies comprising wild-type Fe polypeptides. For example, antibodies comprising modified Fc polypeptides can have altered serum half-life, altered stability, altered susceptibility to cellular enzymes, and/or altered effector function (e.g., as assayed in an NK-dependent or macrophage-dependent assay).

In some embodiments, an Fe polypeptide in an antibody described herein can include amino acid substitutions that modulate effector function. In certain embodiments, an Fe polypeptide in an antibody described herein can include amino acid substitutions that reduce or eliminate effector function.

In some embodiments, an Fe polypeptide includes one or more modifications that alter (relative to a wild-type Fe polypeptide) the Ratio of Affinities of the modified Fe polypeptide to an activating FcγR (such as FcγRIIA or FcγRIIIA) relative to an inhibiting FcγR (such as FcγRIIB):

$$Ratio of Affinities = \frac{Wild - Type to Variant Change in Affinity to {FcyR}_{Activiating}}{Wild - Type to Variant Change in Affinity to {FcyR}_{Inhibiting}} .$$

Where a modified Fc polypeptide has a Ratio of Affinities greater than 1, an antibody herein may have particular use in providing a therapeutic or prophylactic treatment of a disease, disorder, or infection, or the amelioration of a symptom thereof, where an enhanced efficacy of effector cell function (e.g., ADCC) mediated by FcγR is desired, e.g., cancer or infectious disease. Where a modified Fe region has a Ratio of Affinities less than 1, an antibody herein may have particular use in providing a therapeutic or prophylactic treatment of a disease or disorder, or the amelioration of a symptom thereof, where a decreased efficacy of effector cell function mediated by FcγR is desired, e.g., autoimmune or inflammatory disorders. Table 3 lists examples of single, double, triple, quadruple, and quintuple amino acid substitutions in an Fe polypeptide that provide a Ratio of Affinities greater than 1 or less than 1 (see e.g., PCT Publication Nos. WO 04/063351; WO 06/088494; WO 07/024249; WO 06/113665; WO 07/021841; WO 07/106707; WO 2008/140603). Amino acid positions are numbered according to EU numbering scheme.

TABLE 3

| Ratio | Single | Double | Triple | Quadruple | Quintuple |
|---|---|---|---|---|---|
| >1 | F243L | F243L & | F243L, P247L & | L234F, F243L, | L235V, |
| | D270E | R292P | N421K | R292P & Y300L | F243L, R292P, |
| | R292G | F243L & | F243L, R292P & | L235I, F243L, | Y300L & |
| | R292P | Y300L | Y300L | R292P & Y300L | P396L |
| | | F243L & | F243L, R292P & | L235Q, F243L, | L235P, F243L, |
| | | P396L | V305I | R292P & Y300L | R292P, Y300L |
| | | D270E & | F243L, R292P & | F243L, P247L, | & P396L |
| | | P396L | P396L | D270E & N421K | F243L, R292P, |
| | | R292P & | F243L, Y300L & | F243L, R255L, | V305I, Y300L |
| | | Y300L | P396L | D270E & P396L | & P396L |
| | | R292P & | P247L, D270E & | F243L, D270E, | |
| | | V305I | N421K | G316D & R416G | |
| | | R292P & | R255L, D270E & | F243L, D270E, | |
| | | P396L | P396L | K392T & P396L | |
| | | Y300L & | D270E, G316D & | F243L, D270E, | |
| | | P396L | R416G | P396L & Q419H | |
| | | P396L & | D270E, K392T & | F243L, R292P, | |
| | | Q419H | P396L | Y300L, & P396L | |
| | | | D270E, P396L & | F243L, R292P, | |
| | | | Q419H | V305I & P396L | |
| | | | V284M, R292L & | P247L, D270E, | |
| | | | K370N | Y300L & N421K | |
| | | | R292P, Y300L & | R255L, D270E, | |
| | | | P396L | R292G & P396L | |
| | | | | R255L, D270E, | |
| | | | | Y300L & P396L | |
| | | | | D270E, G316D, | |
| | | | | P396L & R416G | |
| <1 | Y300L | F243L & | F243L, R292P & | | |
| | P396L | P396L | V305I | | |
| | | P247L & | | | |
| | | N421K | | | |
| | | R255L & | | | |
| | | P396L | | | |
| | | R292P & | | | |
| | | V305I | | | |
| | | K392T & | | | |
| | | P396L | | | |
| | | P396L & | | | |
| | | Q419H | | | |

### III. ANTIBODIES THAT COMPETITIVELY BIND WITH AN ANTI-CEACAM6 ANTIBODY

Also provided herein are antibodies that competitively bind, or are capable of competitively binding (e.g., competitor antibodies), with one or more CEACAM6 antibodies described herein. In certain instances, an antibody (e.g., competitor antibody) may be considered to compete for binding to CEACAM6 when the competitor binds to the same general region of CEACAM6 as an antibody described herein. In certain instances, an antibody (e.g., competitor antibody) may be considered to compete for binding to CEACAM6 when the competitor binds to the exact same region of CEACAM6 as an antibody described herein (e.g., exact same peptide (linear epitope) or exact same surface amino acids (conformational epitope)). In certain instances, an antibody (e.g., competitor antibody) may be considered capable of competing for binding to CEACAM6 when the competitor binds to the same general region of CEACAM6 as an antibody described herein (e.g., extracellular region or leucine-rich binding domain) under suitable assay conditions. In certain instances, an antibody (e.g., competitor agent) may be considered capable of competing for binding to CEACAM6 when the competitor binds to the exact same region of CEACAM6 as an antibody described herein (e.g., exact same peptide (linear epitope) or exact same surface amino acids (conformational epitope)) under suitable assay conditions.

In certain instances, an antibody (e.g., competitor antibody) may be considered to compete for binding to CEACAM6 when the competitor blocks the binding of one or more antibodies described herein to CEACAM6, for example, under suitable assay conditions. Whether a competitor blocks the binding of one or more antibodies described herein to CEACAM6 may be determined using a suitable competition assay or blocking assay, such as, for example, a blocking assay as described in herein. A competitor antibody may block binding of one or more antibodies described herein to CEACAM6 in a competition or blocking assay by 50% or more (e.g., 55%, 60%, 65%, 70%, 75%, 80%, 85%, 90%, 95% or more, or 100%), and conversely, one or more antibodies described herein may block binding of the competitor antibody to CEACAM6 in a competition or blocking assay by about 50% or more (e.g., e.g., 55%, 60%, 65%, 70%, 75%, 80%, 85%, 90%, 95% or more, or 100%).

In certain instances, an antibody (e.g., competitor antibody) may be considered to compete for binding to CEACAM6 when the competitor binds to CEACAM6 with a similar affinity as one or more antibodies described herein, for example, under suitable assay conditions. In some embodiments, an antibody (i.e., competitor antibody) is considered to compete for binding to CEACAM6 when the competitor binds to CEACAM6 with an affinity that is at least about 50% (e.g., 55%, 60%, 65%, 70%, 75%, 80%, 85%, 90%, 95%, or 100%) of the affinity of one or more antibodies described herein.

Also provided herein are antibodies that bind to, or are capable of binding to, the same epitope as one or more antibodies described herein. In particular, provided herein are antibodies that compete with one or more antibodies described herein for binding to the same epitope (e.g., same peptide (linear epitope) or same surface amino acids (conformational epitope)) on CEACAM6. Such antibodies that bind the same epitope may be referred to as epitope competitors.

### IV. POLYCLONAL AND MONOCLONAL ANTIBODIES

Polyclonal antibodies may be raised in animals (vertebrate or invertebrates, including mammals, birds and fish, including cartilaginous fish) by multiple subcutaneous (se) or intraperitoneal (ip) injections of a relevant antigen and an adjuvant. It may be useful to conjugate the relevant antigen to a protein or other carrier that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups. Non-protein carriers (e.g., colloidal gold) also may be used for antibody production.

Animals can be immunized against the antigen, immunogenic conjugates, or derivatives by combining, e.g., 100 μg or 5 μg of the protein or conjugate (for rabbits or mice, respectively) with three volumes of Freund's complete adjuvant and injecting the solution intradermally at multiple sites. One month later the animals are boosted with one-fifth to one-tenth of the original amount of peptide or conjugate in Freund's complete adjuvant by subcutaneous injection at multiple sites. Seven to 14 days later the animals are bled and the serum is assayed for antibody titer. Animals are boosted until the titer plateaus. Often, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using a hybridoma, e.g., the hybridoma method first described by Kohler et al., Nature, 256:495 (1975), or may be made by other methods such as recombinant DNA methods (see, e.g., U.S. Pat. No. 4,816,567). In the hybridoma method, a mouse or other appropriate host animal, such as a hamster or macaque monkey, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (see, e.g., Goding, Monoclonal Antibodies: Principles and Practice, pp. 59-103 (Academic Press, 1986)).

The hybridoma cells thus prepared are seeded and grown in a suitable culture medium that may contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred mycloma cells are those that fuse efficiently, support stable high-level production of antibody by the selected antibody-producing cells, and are sensitive to a medium such as HAT medium. Among these, preferred myeloma cell lines are murine myeloma lines, such as SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. The binding specificity of monoclonal antibodies produced by hybridoma cells may be determined by immunoprecipitation, by an in vitro binding assay, such as radioimmunoassay (RIA) or enzyme-linked immunoabsorbant assay (ELISA), or by flow cytometric analysis of cells expressing the membrane antigen. The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (see, e.g., Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of the monoclonal antibodies). Alternatively, cDNA may be prepared from mRNA and the cDNA then subjected to DNA sequencing. The hybridoma cells serve as a preferred source of such genomic DNA or RNA for cDNA preparation. Once isolated, the DNA may be placed into expression vectors, which are well known in the art, and which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells.

### V. HUMANIZATION AND AMINO ACID VARIANTS

General methods for humanization of antibodies are described, for example, in U.S. Pat. Nos. 5,861,155, 6,479,284, 6,407,213, 6,639,055, 6,500,931, 5,530,101, 5,585,089, 5,693,761, 5,693,762, 6,180,370, 5,714,350, 6,350,861, 5,777,085, 5,834,597, 5,882,644, 5,932,448, 6,013,256, 6,129,914, 6,210,671, 6,329,511, 5,225,539, 6,548,640, and 5,624,821. In certain embodiments, it may be desirable to generate amino acid sequence variants of these humanized antibodies, particularly where these improve the binding affinity or other biological properties (e.g., half-life) of the antibody.

In some embodiments, the antibody is a humanized antibody, i.e., an antibody that retains the reactivity of a non-human antibody while being less immunogenic in humans. This can be achieved, for instance, by retaining the non-human CDR regions and replacing the remaining parts of the antibody with their human counterparts. See, e.g., Morrison et al., PNAS USA, 81:6851-6855 (1984); Morrison and Oi, Adv. Immunol., 44:65-92 (1988); Verhoeyen et al., Science, 239:1534-1536 (1988); Padlan, Molec. Immun., 28:489-498 (1991); Padlan, Molec. Immun., 31(3):169-217 (1994). Techniques for humanizing antibodies are well known in the art and are described in e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534. Humanized antibodies are further described in, e.g., Winter and Milstein (1991) Nature 349:293. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well-known procedures from a variety of human cells. The CDRs for producing the immunoglobulins of the present disclosure can be similarly derived from monoclonal antibodies capable of specifically binding to CEACAM6.

Amino acid sequence variants of the antibody can be prepared by introducing appropriate nucleotide changes into the antibody DNA, or by peptide synthesis. Such variants include, for example, deletions from, and/or insertions into and/or substitutions of, residues within the amino acid sequences of the antibodies for the examples herein. Any combination of deletion, insertion, and substitution is made to arrive at the final construct, provided that the final construct possesses the desired characteristics. The amino acid changes also may alter post-translational processes of the humanized or variant antibody, such as changing the number or position of glycosylation sites.

One method for identification of certain residues or regions of the antibody that are preferred locations for mutagenesis is called “alanine scanning mutagenesis,” as described by, e.g., Cunningham and Wells, Science, 244:1081-1085 (1989). Here, a residue or group of target residues are identified (e.g., charged residues such as Arg, Asp, His, Lys, and Glu) and replaced by a neutral or negatively charged amino acid (most preferably Ala or poly-Ala) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an N-terminal methionyl residue or the antibody fused to an epitope tag. Other insertional variants include the fusion of an enzyme or a polypeptide that increases the serum half-life of the antibody to the N- or C-terminus of the antibody.

Another type of variant is an amino acid substitution variant. These variants have at least one amino acid residue removed from the antibody molecule and a different residue inserted in its place. The sites of greatest interest for substitutional mutagenesis include the hypervariable regions, but FR alterations are also contemplated. Conservative substitutions are preferred, but more substantial changes may be introduced and the products may be screened. Examples of substitutions are listed below:
(1) Ala (A): Val; Len; Ile; Val
(2) Arg €: Lys; Gln; Asn; Lys
(3) Asn (N): Gln; His; Asp, Lys; Gln; Arg
(4) Asp (D): Glu; Asn
(5) Cys €: Ser; Ala
(6) Gln (Q): Asn; Glu
(7) Glu €: Asp; Gln
(8) Gly (G): Ala
(9) His (H): Asn; Gln; Lys; Arg
(10) Ile (I): Len; Val; Met; Ala; Leu; Phe; Norleucine
(11) Leu (L): Norleucine; Ile; Val; Ile; Met; Ala; Phe
(12) Lys (K): Arg; Gln; Asn
(13) Met (M): Leu; Phe; Ile
(14) Phe (F): Len; Val; Ile; Ala; Tyr
(15) Pro (P): Ala
(16) Ser (S): Thr
(17) Thr (T): Ser
(18) Trp (W): Tyr; Phe
(19) Tyr (Y): Trp; Phe; Thr; Ser
(20) Val (V): Ile; Leu; Met; Phe; Ala; Norleucine

Substantial modifications in the biological properties of an antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val, Leu, Ile;
(2) neutral hydrophilic: Cys, Ser, Thr;
(3) acidic: Asp, Glu;
(4) basic: Asn, Gln, His, Lys, Arg;
(5) residues that influence chain orientation: Gly, Pro; and
(6) aromatic: Trp, Tyr, Phe

Non-conservative substitutions will entail exchanging a member of one of the above classes for another class.

Any cysteine residue not involved in maintaining the proper conformation of the antibody also may be substituted, to improve the oxidative stability of the molecule and prevent aberrant crosslinking. Conversely, cysteine bond(s) may be added to the antibody to improve its stability (particularly where the antibody is an antigen-binding fragment such as an Fv fragment).

One type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino acid substitutions at each site. The antibody variants thus generated can be displayed in the monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis can be performed to identify hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antibody alters the original glycosylation pattern of the antibody. By altering is meant deleting one of more carbohydrate moieties found in the antibody, and/or adding one or more glycosylation sites that are not present in the antibody. Glycosylation of antibodies is typically either N-linked and/or O-linked. N-linked refers to the attachment of the carbohydrate moiety to the side chain of an asparagine residue. The tripeptide sequences asparagine-X-serine and asparagine-X-threonine, where X is any amino acid except proline, are the most common recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Addition of glycosylation sites to the antibody can be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

### VI. OTHER MODIFICATIONS

Other modifications of an antibody are contemplated. For example, technology herein also pertains to immunoconjugates comprising an antibody described herein conjugated to a cytotoxic agent such as a toxin (e.g., an enzymatically active toxin of bacterial, fungal, plant or animal origin, or fragments thereof), or a radioactive isotope (for example, a radioconjugate), or a cytotoxic drug. Such conjugates are sometimes referred to as “antibody-drug conjugates” or “ADC.” Conjugates can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis-(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

In some of any embodiments, any of the antibodies or antigen-binding fragments thereof disclosed herein may be formulated as immunoliposomes. Liposomes containing an antibody are prepared by methods know in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. For example, liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody provided herein can be conjugated to the liposomes as described in Martin et al., J. Biol. Chem. 257:286-288 (1982) via a disulfide interchange reaction. Another active ingredient is optionally contained within the liposome.

Enzymes or other polypeptides can be covalently bound to an antibody by techniques well known in the art such as the use of the heterobifunctional cross-linking reagents discussed above. In some embodiments, fusion proteins comprising at least the antigen-binding region of an antibody provided herein linked to at least a functionally active portion of an enzyme can be constructed using recombinant DNA techniques well known in the art (see, e.g., Neuberger et al., Nature 312:604-608 (1984)).

In certain embodiments, it may be desirable to use an antigen-binding fragment, rather than an intact antibody, to increase penetration of target tissues and cells, for example. In such instances, it may be desirable to modify the antigen-binding fragment to increase its serum half-life. This may be achieved, for example, by incorporation of a salvage receptor binding epitope into the antigen-binding fragment (e.g., by mutation of the appropriate region in the antigen-binding fragment or by incorporating the epitope into a peptide tag that is then fused to the antigen-binding fragment at either end or in the middle, e.g., by DNA or peptide synthesis; see, e.g., WO96/32478 published Oct. 17, 1996).

In some embodiments, any of the antibodies or antigen fragments thereof disclosed herein are conjugated or hybridized to an oligonucleotide. In some embodiments, the oligonucleotide includes a sample barcode sequence, a binding site for a primer and an anchor. In some embodiments, the oligonucleotide can be conjugated or hybridized to any of the detectable markers or labels disclosed herein. In some embodiments, the oligonucleotide is a polymeric sequence. In some embodiments, the terms “oligonucleotide” and “polynucleotide” are used interchangeably to refer to a single-stranded multimer of nucleotides from about 2 to about 500 nucleotides in length. In some embodiments, any of the oligonucleotides described herein can be synthetic, made enzymatically (e.g., via polymerization), or using a “split-pool” method. In some embodiments, any of the oligonucleotides described herein can include ribonucleotide monomers (i.e., can be oligoribonucleotides) and/or deoxyribonucleotide monomers (i.e., oligodeoxyribonucleotides). In some embodiments, any of the oligonucleotides described herein can include a combination of both deoxyribonucleotide monomers and ribonucleotide monomers in the oligonucleotide (e.g., random or ordered combination of deoxyribonucleotide monomers and ribonucleotide monomers). In some embodiments, the oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length. In some embodiments, any of the oligonucleotides described herein can include one or more functional moieties that are attached (e.g., covalently or non-covalently) to another structure. In some embodiments, any of the oligonucleotides described herein can include one or more detectable labels (e.g., a radioisotope or fluorophore). In some embodiments, the anchor is a defined polymer, e.g., a polynucleotide or oligonucleotide sequence, which is designed to hybridize to a complementary oligonucleotide sequence. In some embodiments, the anchor is designed for the purpose of generating a double stranded construct oligonucleotide sequence. In some embodiments, the anchor is positioned at the 3′ end of the construct oligonucleotide sequence. In other embodiments, the anchor is positioned at the 5′ end of the construct oligonucleotide sequence. Each anchor is specific for its intended complementary sequence.

In some embodiments, the sample barcode sequence is a polymer, e.g., a polynucleotide, which when it is a functional element, is specific for a single ligand. In some embodiments, the sample barcode sequence can be used for identifying a particular cell or substrate, e.g., Drop-seq microbead. In some embodiments, the sample barcode sequence can be formed of a defined sequence of DNA, RNA, modified bases or combinations of these bases, as well as any other polymer defined above. In some embodiments, the sample barcode sequence is about 2 to 4 monomeric components, e.g., nucleotide bases, in length. In other embodiments, the barcode is at least about 1 to 100 monomeric components, e.g., nucleotides, in length. Thus in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In some embodiments, the sample barcode sequence is a particular barcode that can be unique relative to other barcodes.

In some of any embodiments, the sample barcode sequences can have a variety of different formats. For example, sample barcode sequences can include polynucleotide barcodes, random nucleic acid and/or amino acid sequences, and synthetic nucleic acid and/or amino acid sequences. A sample barcode sequence can be attached to an analyte or to another moiety or structure in a reversible or irreversible manner. A sample barcode sequences can be added to, for example, a fragment of a deoxyribonucleic acid (DNA) or ribonucleic acid (RNA) sample before or during sequencing of the sample. Sample barcode sequences can allow for identification and/or quantification of individual sequencing-reads (e.g., a barcode can be or can include a unique molecular identifier or “UMI”).

Sample barcode sequences can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

In some embodiments, the binding site for a primer is a functional component of the oligonucleotide which itself is an oligonucleotide or polynucleotide sequence that provides an annealing site for amplification of the oligonucleotide. The binding site for a primer can be formed of polymers of DNA, RNA, PNA, modified bases or combinations of these bases, or polyamides, etc. In some embodiments, the binding site for a primer is about 10 of such monomeric components, e.g., nucleotide bases, in length. In other embodiments, the binding site for a primer is at least about 5 to 100 monomeric components, e.g., nucleotides, in length. Thus in various embodiments, the binding site for a primer is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In certain embodiments, the binding site for a primer can be a generic sequence suitable as a annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependent amplification (I), ramification amplification method and RNA-seq. Methods for conjugating or hybridizing an oligonucleotide can be performed in a manner set forth in WO/2018/144813, WO/2016/018960, WO/2018/089438, WO/2014/182528, WO/2018/026873, WO/2021/188838.

In some embodiments, a modification can optionally be introduced into the antibodies (e.g., within the polypeptide chain or at either the N- or C-terminal), e.g., to extend in vivo half-life, such as PEGylation or incorporation of long-chain polyethylene glycol polymers (PEG). Introduction of PEG or long chain polymers of PEG increases the effective molecular weight of the polypeptides, for example, to prevent rapid filtration into the urine. In some embodiments, a lysine residue in the sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG are known in the art (described, for example, in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which are incorporated herein by reference.

Covalent modifications of an antibody are also included within the scope of this technology. For example, modifications may be made by chemical synthesis or by enzymatic or chemical cleavage of an antibody. Other types of covalent modifications of an antibody are introduced into the molecule by reacting targeted amino acid residues of the antibody with an organic derivatizing agent that is capable of reacting with selected side chains or the N- or C-terminal residues. Example covalent modifications of polypeptides are described in U.S. Pat. No. 5,534,615, specifically incorporated herein by reference. A preferred type of covalent modification of the antibody comprises linking the antibody to one of a variety of non-proteinaceous polymers, e.g., polyethylene glycol, polypropylene glycol, or polyoxyalkylenes, in the manner set forth in, e.g., U.S. Pat. Nos. 4,640,835; 4,496,689; 4,301,144; 4,670,417; 4,791,192 or 4,179,337.

### VII. Nucleic Acids, Vectors, Host Cells, and Recombinant Methods

The disclosure also provides isolated nucleic acids encoding an antibody, vectors and host cells comprising the nucleic acid, and recombinant techniques for the production of the antibody. A nucleic acid herein may include one or more subsequences, each referred to as a polynucleotide.

Provided herein are nucleic acids (e.g., isolated nucleic acids) comprising a nucleotide sequence that encodes an antibody, or fragment thereof. In some embodiments, a nucleic acid encodes an immunoglobulin heavy chain variable domain of an antibody provided herein. In some embodiments, a nucleic acid encodes an immunoglobulin light chain variable domain of an antibody provided herein. In some embodiments, a nucleic acid encodes an immunoglobulin heavy chain variable domain and an immunoglobulin light chain variable domain of an antibody provided herein. In some embodiments, a nucleic acid comprises a nucleotide sequence that encodes an amino acid sequence of any one of SEQ ID Nos: 1-23. In some embodiments, a nucleic acid comprises a nucleotide sequence that encodes an immunoglobulin heavy chain comprising SEQ ID NO: 4 or a sequence having at least 90% sequence identity to the sequence set forth in SEQ ID NO: 4, or an immunoglobulin light chain comprising SEQ ID NO: 5 or a sequence having at least 90% sequence identity to the sequence set forth in SEQ ID NO: 5. In another embodiment, a nucleic acid comprises a nucleotide sequence that encodes an immunoglobulin heavy chain comprising SEQ ID NO: 4 or a sequence having at least 90% sequence identity to the sequence set forth in SEQ ID NO: 4, and an immunoglobulin light chain comprising SEQ ID NO: 5 or a sequence having at least 90% sequence identity to the sequence set forth in SEQ ID NO: 5. In some embodiments, a nucleic acid comprises a nucleotide sequence that encodes an immunoglobulin heavy chain comprising SEQ ID NO: 2 or a sequence having at least 90% sequence identity to the sequence set forth in SEQ ID NO: 2, or an immunoglobulin light chain comprising SEQ ID NO: 3 or a sequence having at least 90% sequence identity to the sequence set forth in SEQ ID NO: 3. In another embodiment, a nucleic acid comprises a nucleotide sequence that encodes an immunoglobulin heavy chain comprising SEQ ID NO: 2 or a sequence having at least 90% sequence identity to the sequence set forth in SEQ ID NO: 2, and an immunoglobulin light chain comprising SEQ ID NO: 3 or a sequence having at least 90% sequence identity to the sequence set forth in SEQ ID NO: 3.

Provided herein is a nucleotide sequence that encodes the immunoglobulin heavy chain variable domain and the immunoglobulin light chain variable domain of the antibody or antigen-binding fragment thereof of any of any of the antibodies or antigen-binding fragments provided herein.

For recombinant production of an antibody, a nucleic acid encoding the antibody may be isolated and inserted into a replicable vector for further cloning (amplification of the DNA) or for expression. In certain instances, an antibody may be produced by homologous recombination. DNA encoding an antibody can be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, and origin of replication, one or more marker genes, an enhancer element, a promoter, and a transcription termination sequence.

Suitable host cells for cloning or expressing DNA in vectors herein can be prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. lichenmformis, Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) can also be suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. A number of other genera, species, and strains are commonly available and useful herein, such as Schizosaccharomyces pombe; Kluyveronmyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of antibodies can also be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori (silk moth) have been identified. A variety of viral strains for transfection are publicly available, e.g., the L-1 variant of Autographa californica NPV and the Bm-5 strain of Bombyx mori NPV, and such viruses may be used as the virus herein according to the present technology, particularly for transfection of Spodoptera frugiperda cells. Plant cell cultures of cotton, corn, potato, soybean, petunia, tomato, and tobacco can also be utilized as hosts.

Suitable host cells for the expression of antibodies also may include vertebrate cells (e.g., mammalian cells). Vertebrate cells may be propagated in culture (tissue culture). Examples of useful mammalian host cell lines include monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse Sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells may be transformed with the above-described expression or cloning vectors for antibody production and cultured in conventional nutrient media modified as appropriate for inducing promoters, selecting transformants, or amplifying the genes encoding the desired sequences. Host cells used to produce antibodies provided herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, antibodies can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies that are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, Bakerbond ABX™ resin (J. T. Baker, Phillipsburg, N.J.) is useful for purification. Other techniques for protein purification, such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between, e.g., about 2.5-4.5, and may be performed at low salt concentrations (e.g., from about 0-0.2 5M salt).

### VIII. PHARMACEUTICAL FORMULATIONS, DOSING, AND ROUTES OF ADMINISTRATION

The present disclosure provides antibodies and related compositions, which may be useful for elimination of—expressing pathogens from the body, for example, and for identification and quantification of the number of CEACAM6-expressing pathogens in biological samples, for example.

In some embodiments, any of the antibodies or antigen-binding fragments thereof may be formulated in a pharmaceutical composition that is useful for a variety of purposes, including the treatment of diseases or disorders. Pharmaceutical compositions comprising one or more antibodies may be administered using a pharmaceutical device to a patient in need thereof, and according to one embodiment of the technology, kits are provided that include such devices. Such devices and kits may be designed for routine administration, including self-administration, of the pharmaceutical compositions herein.

Therapeutic formulations of an antibody may be prepared for storage by mixing the agent or antibody having the desired degree of purity with optional physiologically acceptable carriers, excipients or stabilizers (Remington's Pharmaceutical Sciences 16^{th} edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

In some embodiments, the disease or disorder is associated with CEACAM6 expression. In some embodiments, the disease or disorder is associated with aberrant CEACAM6 expression. In some embodiments, the disease or disorder is associated with Natural Killer (NK), alpha beta T cells, gamma delta T cells, CD8+ T cells, monocytes, or dendritic cells. In some embodiments, the disease or disorder is associated with Natural Killer (NK) cells. In some embodiments, the disease or disorder is associated with alpha beta T cells. In some embodiments, the disease or disorder is associated with gamma delta T cells. In some embodiments, the disease or disorder is associated with CD8+ T cells. In some embodiments, the disease or disorder is associated with monocytes. In some embodiments, the disease or disorder is associated with dendritic cells.

In some embodiments, the disease or disorder is a cancer, an infectious disease, or an autoimmune disorder.

In some embodiments, the disease or disorder is a cancer. In some embodiments, the cancer is, e.g., metastatic melanoma, a solid tumor, bladder cancer, head and neck squamous cell carcinoma, hepatocellular carcinoma, hepatic metastasis of colonic origin, papillary thyroid carcinoma, acute myeloid leukemia, or asymptomatic myeloma.

In some embodiments, the disease or disorder is an infectious disease. In some embodiments, the infectious disease is, e.g., human immunodeficiency virus (HIV), chronic hepatitis C, cytomegalovirus, or hantavirus.

In some embodiments, the disease or disorder is an autoimmune disorder. In some embodiments, the autoimmune disorder is, e.g., Crohn's disease, multiple sclerosis, systemic sclerosis, ocular myasthenia gravis, psoriasis or rheumatoid arthritis.

In some embodiments, any of the antibodies or antigen-binding fragments thereof described herein can be used to decrease the production of androgenic hormones in prostate cancer cells.

In some embodiments, any of the antibodies or antigen-binding fragments thereof described herein can be used to inhibit or reduce cleavage of coronavirus spike glycoproteins. In some embodiments, any of the antibodies or antigen-binding fragments thereof described herein can be used to inhibit or reduce viral uptake into a host cell.

Formulations herein may also contain more than one active compound as necessary for the particular indication being treated, preferably those with complementary activities that do not adversely affect each other. Such molecules are suitably present in combination in amounts that are effective for the purpose intended.

Formulations for in vivo administration generally are sterile. This may be accomplished for instance by filtration through sterile filtration membranes, for example.

Sustained-release preparations may be prepared. Suitable examples of sustained-release preparations include semipermeable matrices of solid hydrophobic polymers containing the agent/antibody, which matrices are in the form of shaped articles, e.g., films, or microcapsule. Examples of sustained-release matrices include polyesters, hydrogels (for example, poly(2-hydroxyethyl-methacrylate), or poly (vinyl alcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid. While polymers such as such as ethylene-vinyl acetate and lactic acid-glycolic acid enable release of molecules for over 100 days, certain hydrogels release proteins for shorter time periods. When encapsulated agents/antibodies remain in the body for a long time, they may denature or aggregate as a result of exposure to moisture at 37 °C, resulting in a loss of biological activity and possible changes in immunogenicity. Rational strategies can be devised for stabilization depending on the mechanism involved. For example, if the aggregation mechanism is discovered to be intermolecular S—S bond formation through thiol-disulfide interchange, stabilization may be achieved by modifying sulfhydryl residues, lyophilizing from acidic solutions, controlling moisture content, using appropriate additives, and developing specific polymer matrix compositions.

For therapeutic applications, antibodies provided herein are administered to a mammal, e.g., a human, in a pharmaceutically acceptable dosage form such as those discussed above, including those that may be administered to a human intravenously as a bolus or by continuous infusion over a period of time, or by intramuscular, intraperitoneal, intra-cerebrospinal, subcutaneous, intra-articular, intrasynovial, intrathecal, oral, topical, or inhalation routes. For the prevention or treatment of disease, the appropriate dosage of agent or antibody will depend on the type of disease to be treated, as defined above, the severity and course of the disease, whether the antibody is administered for preventative or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to about 50 mg/kg (e.g., 0.1-20 mg/kg) of antibody may be an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Detection methods using the antibody to determine TMPRSS2 levels in bodily fluids or tissues may be used to optimize patient exposure to the therapeutic antibody.

In some embodiments, a composition comprising an antibody herein can be administered as a monotherapy, and in some embodiments, the composition comprising the antibody can be administered as part of a combination therapy. In some cases, the effectiveness of the antibody in preventing or treating diseases may be improved by administering the antibody serially or in combination with another drug that is effective for those purposes, such as a chemotherapeutic drug for treatment of cancer or a microbial infection. In other cases, the antibody may serve to enhance or sensitize cells to chemotherapeutic treatment, thus permitting efficacy at lower doses and with lower toxicity. Certain combination therapies include, in addition to administration of the composition comprising an antibody that reduces the number of—expressing cells, delivering a second therapeutic regimen selected from the group consisting of a chemotherapeutic agent, radiation therapy, surgery, and a combination of any of the foregoing. Such other agents may be present in the composition being administered or may be administered separately. Also, the antibody may be suitably administered serially or in combination with the other agent or modality, e.g., chemotherapeutic drug or radiation for treatment of cancer, infection, and the like, or an immunosuppressive drug.

### IX. RESEARCH AND DIAGNOSTIC

Also provided herein are diagnostic reagents comprising an antibody described herein. For example, antibodies provided herein may be used to detect and/or purify CEACAM6 from bodily fluid(s) or tissues. Also provided herein are methods for detecting CEACAM6. For example, a method may comprise contacting a sample (e.g., a biological sample known or suspected to contain) with an antibody provided herein, and, if the sample contains CEACAM6, detecting CEACAM6:antibody complexes. Also provided herein are reagents comprising an antibody described herein and methods for detecting for research purposes.

Any of the antibodies or antigen-binding fragments disclosed herein can be useful in diagnostic assays for detecting its presence in specific cells, tissues, or bodily fluids. Such diagnostic methods may be useful in diagnosis, e.g., of a hyperproliferative disease or disorder. Thus, clinical diagnostic uses as well as research uses are comprehended herein. In some embodiments, an antibody comprises a detectable marker or label. In some embodiments, an antibody is conjugated to a detectable marker or label. For example, for research and diagnostic applications, an antibody may be labeled with a detectable moiety. Numerous labels are available which are generally grouped into the following categories:
(a) Radioisotopes, such as 35S, 14C, 125I, 3H, and 131I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.
(b) Fluorescent labels such as rare earth chelates (europium chelates) or fluorescein and its derivatives, rhodamine and its derivatives, dansyl, Lissamine, phycoerythrin, Texas Red and Brilliant Violet™ are available. The fluorescent labels can be conjugated to the antibody using the techniques disclosed in Current Protocols in Immunology, supra, for example. Fluorescence can be quantified using a flow cytometer, imaging microscope or fluorimeter.

Various enzyme-substrate labels are available. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemilluminometer, for example) or donates energy to a fluorescent acceptor. Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone & H. Van Vunakis), Academic press, New York, 73:147-166 (1981).

Examples of enzyme-substrate combinations include, for example:
(i) Horseradish peroxidase (HRP) with hydrogen peroxidase as a substrate, where the hydrogen peroxidase oxidizes a dye precursor (e.g., orthophenylene diamine (OPD) or 3,3′,5,5′-tetramethyl benzidine hydrochloride (TMB));
(ii) alkaline phosphatase (AP) with para-Nitrophenyl phosphate as chromogenic substrate; and (iii) β-D-galactosidase (β-D-Gal) with a chromogenic substrate (e.g., p-nitrophenyl-β-D-galactosidase) or fluorogenic substrate 4-methylumbelliferyl-β-D-galactosidase.

In certain instances, the label is indirectly conjugated with the agent or antibody. The skilled artisan will be aware of various techniques for achieving this. For example, an antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In some embodiments, and antibody or antigen-binding fragments thereof need not be labeled, and the presence thereof can be detected, e.g., using a labeled antibody which binds to an antibody.

In some embodiments, an antibody herein is immobilized on a solid support or substrate. In some embodiments, an antibody herein is non-diffusively immobilized on a solid support (e.g., the antibody does not detach from the solid support). A solid support or substrate can be any physically separable solid to which an antibody can be directly or indirectly attached including, but not limited to, surfaces provided by microarrays and wells, and particles such as beads (e.g., paramagnetic beads, magnetic beads, microbeads, nanobeads), microparticles, and nanoparticles. Solid supports also can include, for example, chips, columns, optical fibers, wipes, filters (e.g., flat surface filters), one or more capillaries, glass and modified or functionalized glass (e.g., controlled-pore glass (CPG)), quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polybutylene, polyurethanes, TEFLON™ polyethylene, polypropylene, polyamide, polyester, polyvinylidenedifluoride (PVDF), and the like), polysaccharides, nylon or nitrocellulose, resins, silica or silica-based materials including silicon, silica gel, and modified silicon, Sephadex®, Sepharose®, carbon, metals (e.g., steel, gold, silver, aluminum, silicon and copper), inorganic glasses, conducting polymers (including polymers such as polypyrrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In some embodiments, the solid support or substrate may be coated using passive or chemically-derivatized coatings with any number of materials, including polymers, such as dextrans, acrylamides, gelatins or agarose. Beads and/or particles may be free or in connection with one another (e.g., sintered). In some embodiments, a solid support or substrate can be a collection of particles. In some embodiments, the particles can comprise silica, and the silica may comprise silica dioxide. In some embodiments the silica can be porous, and in certain embodiments the silica can be non-porous. In some embodiments, the particles further comprise an agent that confers a paramagnetic property to the particles. In certain embodiments, the agent comprises a metal, and in certain embodiments the agent is a metal oxide, (e.g., iron or iron oxides, where the iron oxide contains a mixture of Fe2+ and Fe3+). An antibody may be linked to a solid support by covalent bonds or by non-covalent interactions and may be linked to a solid support directly or indirectly (e.g., via an intermediary agent such as a spacer molecule or biotin).

Antibodies and antigen-binding fragments thereof provided herein may be employed in any known assay method, such as flow cytometry, immunohistochemistry, immunofluorescence, mass cytometry (e.g., Cytof instrument), competitive binding assays, direct and indirect sandwich assays, and immunoprecipitation assays. Zola, Monoclonal Antibodies: A Manual of Techniques, pp. 147-158 (CRC Press, Inc. 1987). Flow cytometry and mass cytometry assays generally involve the use of a single primary antibody to specifically identify the presence of the target molecule expressed on the surface of a dispersed suspension of individual cells. The dispersed cells are typically obtained from a biological fluid sample, e.g., blood, but may also be obtained from a dispersion of single cells prepared from a solid tissue sample such as spleen or tumor biopsy. The primary antibody may be directly conjugated with a detectable moiety, e.g., a fluorophore such as phycoerythrin for flow cytometry or a heavy metal chelate for mass cytometry. Alternatively, the primary antibody may be unlabeled or labeled with an undetectable tag such as biotin, and the primary antibody is then detected by a detectably labeled secondary antibody that specifically recognizes the primary antibody itself or the tag on the primary antibody. The labeled cells are then analyzed in an instrument capable of single cell detection, e.g., flow cytometer, mass cytometer, fluorescence microscope or brightfield light microscope, to identify those individual cells in the dispersed population or tissue sample that express the target recognized by the primary antibody. Detailed description of the technological basis and practical application of flow cytometry principles may be found in, e.g., Shapiro, Practical Flow Cytometry, 4^{th} Edition, Wiley, 2003.

Sandwich assays involve the use of two antibodies, each capable of binding to a different immunogenic portion, or epitope, of the protein that is detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. In a cell ELISA, the target cell population may be attached to the solid support using antibodies first attached to the support and that recognize different cell surface proteins. These first antibodies capture the cells to the support. CEACAM6 on the surface of the cells can then be detected by adding any of the anti-CEACAM6 antibodies or antigen-binding fragments thereof described herein to the captured cells and detecting the amount of the anti-CEACAM6 antibody or antigen-binding fragment thereof attached to the cells. In certain instances, fixed and permeabilized cells may be used, and in such instances, both surface CEACAM6 and intracellular CEACAM6 may be detected.

In some embodiments, any of the antibodies or antigen-binding fragments thereof provided herein are formulated for immunohistochemical analysis. In some embodiments, immunohistochemical analysis includes the use of samples. In some embodiments, immunohistochemical analysis includes the use of blood and/or tissue samples. In some embodiments, the sample may be fresh or frozen or may be embedded in paraffin and fixed with a preservative such as formalin. In some embodiments, the sample is a formalin-fixed paraffin-embedded (FFPE) sample. In some embodiments, the FFPE sample is saturated with formalin (i.e. formaldehyde) and then embedded in a block of paraffin wax. In some embodiments, the FFPE sample is stable at room temperature. In some embodiments, all of the structures in the FFPE sample are preserved. In some embodiments, the intracellular and surface proteins in the FFPE sample are preserved. In some embodiments, the mRNA in the FFPE sample is preserved. In some embodiments, the mRNA, intracellular and surface proteins in the FFPE sample are preserved. In some embodiments, the surface proteins in the FFPE sample are denatured.

In some embodiments, any of the anti-CEACAM6 antibodies or antigen-binding fragments thereof provided herein are capable of detecting CEACAM6 in a formalin-fixed paraffin-embedded sample. In some embodiments, any of the anti-CEACAM6 antibodies or antigen-binding fragments thereof provided herein are capable of detecting CEACAM6 on the surface of a cell in a formalin-fixed paraffin-embedded sample. In some embodiments, any of the anti-CEACAM6 antibodies or antigen-binding fragments thereof provided herein are capable of detecting intracellular CEACAM6 in a formalin-fixed paraffin-embedded sample. In some embodiments, any of the anti-CEACAM6 antibodies or antigen-binding fragments thereof provided herein are capable of detecting intracellular CEACAM6, and CEACAM6 on the surface of a formalin-fixed paraffin-embedded sample.

In some embodiments, the sample is a fresh sample that has been frozen. In some embodiments, the sample is a fresh sample that has been cryogenically frozen. In some embodiments, the sample is flash frozen. In some embodiments, the sample if flash frozen and stored at 80 °C. In some embodiments, all of the structures in the flash frozen sample are preserved. In some embodiments, the intracellular and surface proteins in the flash frozen sample are preserved. In some embodiments, the mRNA in the flash frozen sample is preserved. In some embodiments, the mRNA, intracellular and surface proteins in the flash frozen sample are preserved. In some embodiments, the surface proteins in the flash frozen sample are denatured.

In some embodiments, any of the anti-CEACAM6 antibodies or antigen-binding fragments thereof provided herein are capable of detecting CEACAM6 in a frozen sample. In some embodiments, any of the anti-CEACAM6 antibodies or antigen-binding fragments thereof provided herein are capable of detecting CEACAM6 on the surface of a frozen sample. In some embodiments, any of the anti-CEACAM6 antibodies or antigen-binding fragments thereof provided herein are capable of detecting intracellular CEACAM6 in a frozen sample. In some embodiments, any of the anti-CEACAM6 antibodies or antigen-binding fragments thereof provided herein are capable of detecting intracellular CEACAM6, and CEACAM6 on the surface of a frozen sample.

The antibodies herein also may be used for in vivo diagnostic assays. Generally, the antibody is labeled with a radionuclide (such as 111In, 99Tc, 14C, 131I, 125I, 3H, 32P, or 35S) so that the bound target molecule can be localized using immunoscintillography.

### X. DETECTION OF CEACAM6

Provided herein are antibodies and methods for detecting CEACAM6. In some embodiments, antibodies and methods are provided for detecting CEACAM6 in a biological sample. In some embodiments, the biological sample is a solid tissue, fluid, or cell. In some embodiments, the CEACAM6 is detected on the surface of the cell. In some embodiments, the CEACAM6 is detected intracellularly. In some embodiments, the detection of CEACAM6 is in vitro. In some embodiments, the detection of CEACAM6 is in vivo.

The solid tissue may comprise solid tissue from one or more of adipose tissue, bladder, bone, brain breast cervix, endothelium, gallbladder, kidney, liver, lung, lymph, ovary, prostate, salivary gland, stomach, testis, thyroid, urethra, uterus, vagina, and vulva. In some embodiments, the fluid comprises one or more of amniotic fluid, bile, blood, breast milk, breast fluid, cerebrospinal fluid, lavage fluid, lymphatic fluid, mucous, plasma, saliva, semen, serum, spinal fluid, sputum, tears, umbilical cord blood, urine, and vaginal fluid.

In some embodiments, the sample comprises immune cells. In some embodiments, the sample comprises a heterogeneous population of immune cells. In some embodiments, the immune cell is selected from B cells, plasmacytoid dendritic cells (pDCs), lymphocytes, leukocytes, T cells, monocytes, macrophages, neutrophils, myeloid dendritic cells (mDCs), innate lymphoid cells, mast cells, cosinophils, basophils, natural killer cells, and peripheral blood mononuclear cells (PBMCs).

In some of any embodiments, any of the antibodies or antigen-binding fragments thereof provided herein can be used in the characterization of single cells by measurement of gene-expression levels and cellular proteins. Among such known single cell sequencing platforms suitable for integration with the antibodies or antigen-binding fragments thereof described herein is the Drop-seq method, including, but not limited to, microfluidic, plate-based, or microwell, Seq-Well™ method and adaptations of the basic protocol, and InDrop™ method. In another embodiment, a single cell sequencing platform suitable for integration with the antibodies or antigen-binding fragments thereof described herein is 10× genomics single cell 3′ solution or single cell V(D)J solution, either run on Chromium controller, or dedicated Chromium single cell controller. Other suitable sequencing methods include Wafergen iCell8™ method, Microwell-seq method, Fluidigm CI™ method and equivalent single cell products. Still other known sequencing protocols useful with the antibodies or antigen-binding fragments thereof described herein include BD Resolve™ single cell analysis platform and ddSeq (from Illumina® Bio-Rad® SureCell™ WTA 3′ Library Prep Kit for the ddSEQ™ System, 2017, Pub. No. 1070-2016-014-B, Illumina Inc., Bio-Rad Laboratories, Inc.). In still other embodiments, the antibodies or antigen-binding fragments thereof described herein are useful with combinatorial indexing based approaches (sci-RNA-seq™ method or SPLiT-seq™ method) and Spatial Transcriptomics, or comparable spatially resolved sequencing approaches. The methods and compositions described herein can also be used as an added layer of information on standard index sorting (FACS) and mRNA-sequencing-based approaches.

In some of any embodiments, any of the antibodies or antigen-binding fragments thereof described herein can be used to detect the presence, absence or amount of the various nucleic acids, proteins, targets, oligonucleotides, amplification products and barcodes described herein.

In some embodiments, the biological sample is from a healthy subject. In some embodiments, the sample is from a subject with a disease or condition. In some embodiments, the detection of CEACAM6 indicates the presence or absence of a disease or disorder. In some embodiments, the disease or disorder is a cancer, an autoimmune disorder, an inflammatory disorder, a neurologic disorder, or an infection. In some embodiments, the cancer is the cancer is acute myeloid leukemia, acute lymphoblastic leukemia, colorectal, ovarian, gynecologic, liver, glioblastoma, Hodgkin lymphoma, chronic lymphocytic leukemia, esophagus, gastric, pancreas, colon, kidney, head and neck, lung and melanoma.

In some embodiments, the disease or disorder is associated with CEACAM6 expression, In some embodiments, the disease or disorder is associated with aberrant CEACAM6 expression. In some embodiments, the disease or disorder is associated with Natural Killer (NK), alpha beta T cells, gamma delta T cells, CD8+ T cells, monocytes, or dendritic cells. In some embodiments, the disease or disorder is associated with Natural Killer (NK) cells. In some embodiments, the disease or disorder is associated with alpha beta T cells. In some embodiments, the disease or disorder is associated with gamma delta T cells. In some embodiments, the disease or disorder is associated with CD8+ T cells. In some embodiments, the disease or disorder is associated with monocytes. In some embodiments, the disease or disorder is associated with dendritic cells. In some of any embodiments, the disease or disorder is chosen from non-viral cancers, virus-associated cancers, cancers associated with HBV infection, cancers associated with Epstein-Barr virus (EBV) infection, cancers associated with polyomavirus infection, erythema nodosum leprosum (ENL), autoimmune diseases, autoimmune inflammation, autoimmune thyroid diseases, B-cell lymphoma, T-cell lymphoma, acute myeloid leukemia, Hodgkin's Disease, acute myelogenous leukemia, acute myelomonocytic leukemia, acute lymphoblastic leukemia, chronic myelogenous leukemia, chronic lymphocytic leukemia, B cell large cell lymphoma, malignant lymphoma, acute leukemia, lymphosarcoma cell leukemia, B-cell leukemias, myelodysplastic syndromes, solid phase cancer, herpes viral infections, and/or rejection of transplanted tissues or organs.

In some embodiments, the disease or disorder is a cancer, an infectious disease, or an autoimmune disorder.

In some embodiments, the disease or disorder is a cancer. In some embodiments, the cancer is metastatic melanoma, a solid tumor, bladder cancer, head and neck squamous cell carcinoma, hepatocellular carcinoma, hepatic metastasis of colonic origin, papillary thyroid carcinoma, acute myeloid leukemia, or asymptomatic myeloma.

In some embodiments, the disease or disorder is an infectious disease. In some embodiments, the infectious disease is human immunodeficiency virus (HIV), chronic hepatitis C, cytomegalovirus, or hantavirus.

In some embodiments, the disease or disorder is an autoimmune disorder. In some embodiments, the autoimmune disorder is Crohn's disease, multiple sclerosis, systemic sclerosis, ocular myasthenia gravis, psoriasis or rheumatoid arthritis. In some embodiments, the autoimmune disorder is Crohn's disease, multiple sclerosis, systemic sclerosis, ocular myasthenia gravis, psoriasis or rheumatoid arthritis.

In some embodiments, any of the antibodies or antigen-binding fragments thereof can be used in generating a nucleic acid molecule comprising all or a portion of the sequence of the oligonucleotide or a complement thereof. In some of any embodiments, the antibody or antigen-binding fragment thereof can be used in a method of associating presence or abundance of CEACAM6 with a location of interest of a tissue sample.

In some embodiments, any of the antibodies or antigen-binding fragments thereof can be used in the construction of a protein library. In some of any embodiments, the construction of a protein library comprises sequencing. In some of any embodiments, the construction of a protein library comprises the use of flow cytometry.

In some of any embodiments, provided herein is a method of detecting CEACAM6, comprising
a) contacting a sample with the antibody or antigen-binding fragment thereof of any of the antibodies or antigen-binding fragments thereof under conditions to bind said antibody or antigen-binding fragment thereof to a CEACAM6 receptor on said sample, wherein the binding generates the production of a receptor/antibody or antigen-binding fragment thereof of complex;
b) detecting the presence of the receptor/antibody or antigen-binding fragment thereof of complexes; c) wherein the detecting comprises the presence or absence of the CEACAM6 receptor on said sample.

In some of any embodiments, provided herein is a method of treating or preventing a disease or disorder associated with CEACAM6 in a subject, comprising: a) contacting a sample known or suspected to contain CEACAM6 with the antibody or antigen-binding fragment thereof any of the antibodies or antigen-binding fragments thereof, b) detecting the presence of complexes comprising CEACAM6 and the antibody or antigen-binding fragment thereof; wherein the presence of the complexes indicates the presence of a disease or disorder; and c) administering to the subject the antibody or antigen-binding fragment thereof of any of the antibodies or antigen-binding fragments thereof.

In some of any embodiments, provided herein is a method of diagnosing a disease or disorder, comprising: a) isolating a sample from a subject, b) incubating the sample with the antibody or antigen-binding fragment thereof of any of any of the antibodies or antigen-binding fragments thereof, for a period of time sufficient to generate CEACM6:anti-CEACAM6 complexes; c) detecting the presence or absence of the CEACAM6:anti-CEACAM6 complexes from the isolated tissue, and d) associating presence or abundance of CEACAM6 with a location of interest of a tissue sample.

In some of any embodiments, the increase of CEACAM6 over a control level in the location of interest of the tissue sample is indicative of a disease or disorder in a subject.

In some of any embodiments, the detection comprises hybridization of a detectable moiety to the antibody or antigen-binding fragment thereof. In some of any embodiments, the sample is contacted with a second antibody. In some of any embodiments, the second antibody is an antibody comprising a detectable moiety. In some of any embodiments, the detectable moiety comprises an oligonucleotide. In some of any embodiments, the detectable moiety comprises a fluorescent label. In some of any embodiments, the measurement comprises sequencing. In some of any embodiments, the detectable moiety comprises immunofluorescence. In some of any embodiments, the sample is a formalin-fixed paraffin-embedded sample. In some of any embodiments, the sample comprises a cell. In some of any embodiments, the sample comprises a tissue sample.

### XI. KITS INCORPORATING ANTI-ANTIBODIES

An antibody herein may be provided in a kit, for example, a packaged combination of reagents in predetermined amounts with instructions for use (e.g., instructions for performing a diagnostic assay; instructions for performing a laboratory assay). In some embodiments, the kit is a diagnostic kit configured to detect CEACAM6 in a sample (e.g., a biological sample). Where the antibody is labeled with a fluorophore, the kit may include an identical isotype negative control irrelevant antibody to control for non-specific binding of the antibody. Where the antibody is labeled with an enzyme, the kit may include substrates and cofactors required by the enzyme (e.g., substrate precursor which provides the detectable chromophore or fluorophore). Additional additives may be included such as stabilizers, buffers (e.g., a block buffer or lysis buffer), and the like. The relative amounts of the various reagents may be varied widely to provide for concentrations in solution of the reagents that substantially optimize the sensitivity of the assay. In certain instances, reagents may be provided as dry powders (e.g., lyophilized powder), including excipients that on dissolution will provide a reagent solution having the appropriate concentration.

### EXAMPLES

### Example 1. Generation Anti-CEACAM6 Antibody Expressing Hybridomas

This Example describes the generation and characterization of hybridomas that secrete monoclonal antibodies that react with CEACAM6.

Briefly, animals were immunized with a CEACAM6 immunogen (recombinant protein amino acids 35-320 numbered relative to SEQ ID NO:1), and hybridomas were formed using standard protocols to fuse myeloma cells with spleens, and lymph node cells were drained and harvested. Successful fusions were selected into HAT medium and cloned into approximately one cell per well in microtiter plates, after which culture supernatants were tested against CEACAM6-expressing cell transfectants by flow cytometry. Wells were selected by assessment of staining profiles and then sub-cultured into larger vessels and sub-cloned. Hybridoma sub-clones were further characterized by flow cytometry using CEACAM6-transfected cells. Candidate clones expressing exemplary anti-CEACAM6 antibodies were selected and screened using various methods, including by flow cytometry against human blood cells divided into distinct subsets (e.g., lymphocytes, monocytes, and the like), and against one or more cell lines generated from diseased and/or infected human cells. The percentage of positive cells in each blood cell subset was quantified as compared to isotype control.

### Example 2. Sequencing of Exemplary Anti-CEACAM6 Antibody Variable Regions

This Example describes the sequencing of exemplary anti-CEACAM6 antibodies generated in Example 1 above.

Cells from anti-CEACAM6 hybridoma cell lines described in Example 1 above were grown in standard mammalian tissue culture media. Total RNA was isolated from hybridoma cells from various clones expressing anti-CEACAM6 monoclonal antibodies using a procedure based on the Rneasy Mini Kit (Qiagen). Hybridoma cells were sequenced for the heavy and light chain VDJ rearrangements. Briefly, RNA was used to generate a first strand of cDNA of the light chain and heavy chain variable domains. Both light chain and heavy chain variable domain cDNAs were amplified by a template switch technique using gene-specific C-primers to enrich the variable gene sequences. Amplified libraries were sequenced using Illumina next generation sequencing on a 500-cycle kit using single end reads from read 1.

Amino acid sequences of the individual variable domains (CDRs and Framework regions), including the CDR1, CDR2, and CDR3 regions, for both the heavy and light chains for two different antibodies (clones) are shown in Table 1 and CDR sets of anti-CEACAM6 antibodies are shown in Table 2.

### Example 3. Detection of CEACAM6 Expressing Cells Using Exemplary Anti-CEACAM Antibodies

This Example describes the ability of exemplary generated anti-CEACAM6 antibodies to detect cells expressing CEACAM6 by flow cytometry and immunohistochemistry (IHC).

In a first experiment, the cross-reactivity of exemplary anti-CEACAM6 antibodies with another CEACAM family was assessed on cells from an immortalized rat colorectal lymphoblast cell line (RBL-1, ATCC CRL-1378) transfected with human CEACAM8, also known as CD66b.

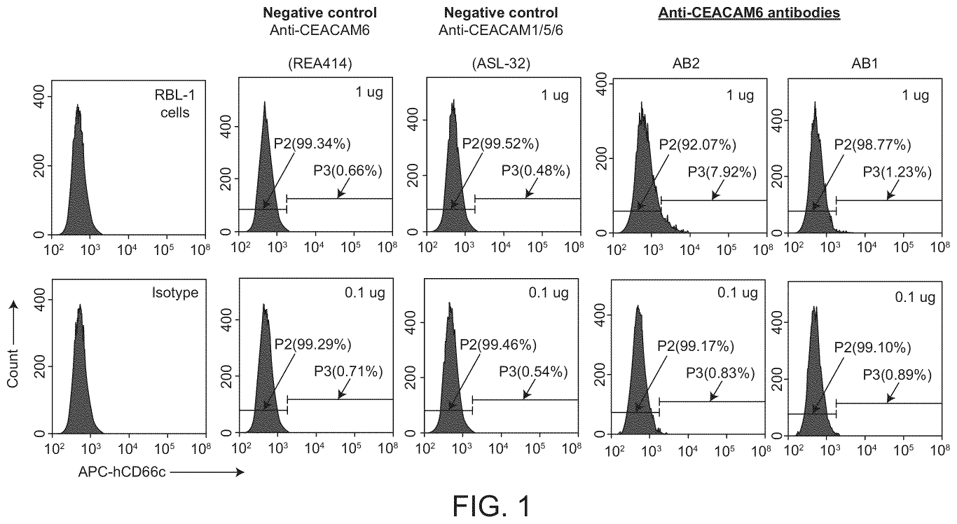

RBL-1 cells were grown in EMEM media supplemented with 10% FBS in T75 culture flask, to about 80% confluency. Cells were suspended in Cell Staining Buffer, and 1 μg or 0.1 ug anti-CEACAM6 antibodies AB1 and AB2 were added and allowed to incubate for 15 minutes. Cells were then washed twice with FACS wash buffer and stained with anti-rat IgG-APC secondary antibody for 15 minutes. Cells were washed with FACS buffer and analyzed on a BD LSRII flow cytometer. Two commercially available anti-CEACAM6 antibodies were used as negative controls (REA414 and ASL-32). As shown in FIG. 1, exemplary tested anti-CEACAM6 antibodies AB1 and AB2 did not show any binding to CEACAM8 and can hence be considered to lack cross-reactivity with CEACAM8.

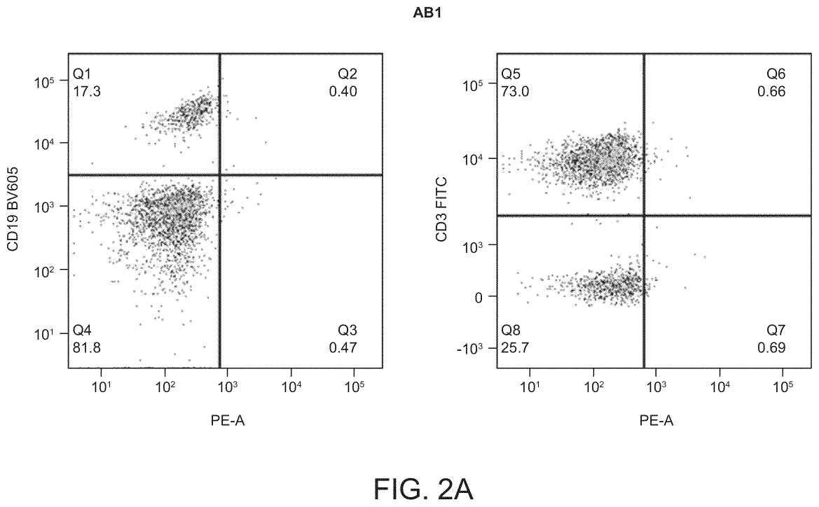

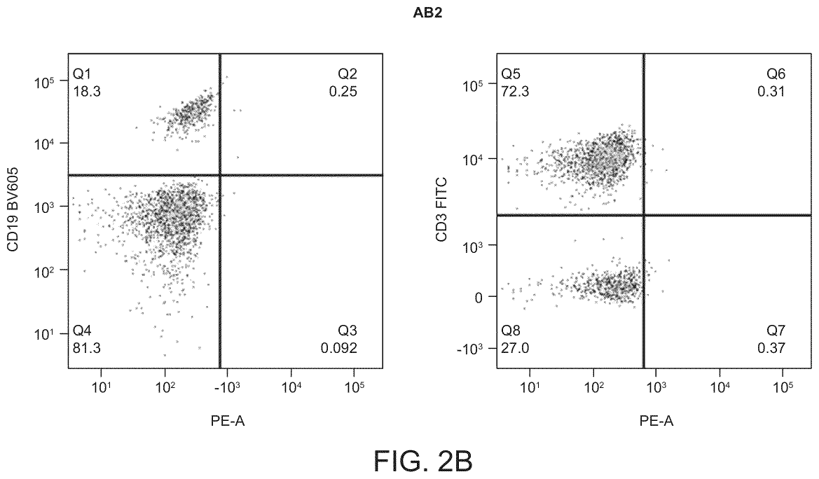

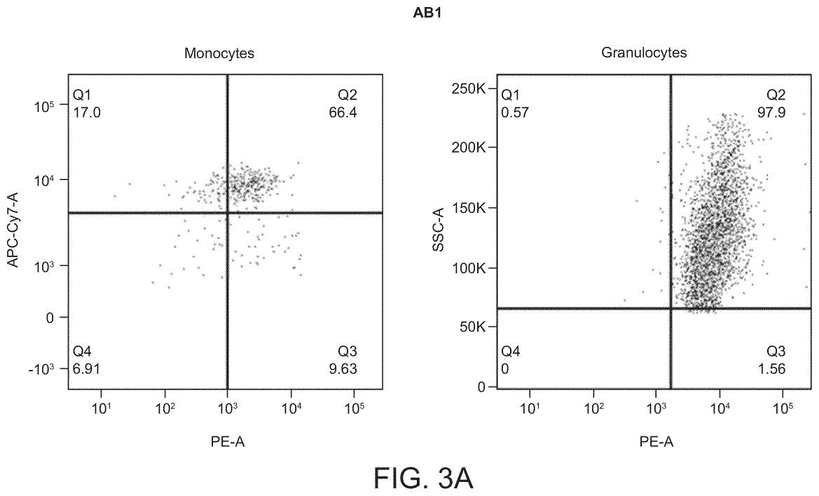

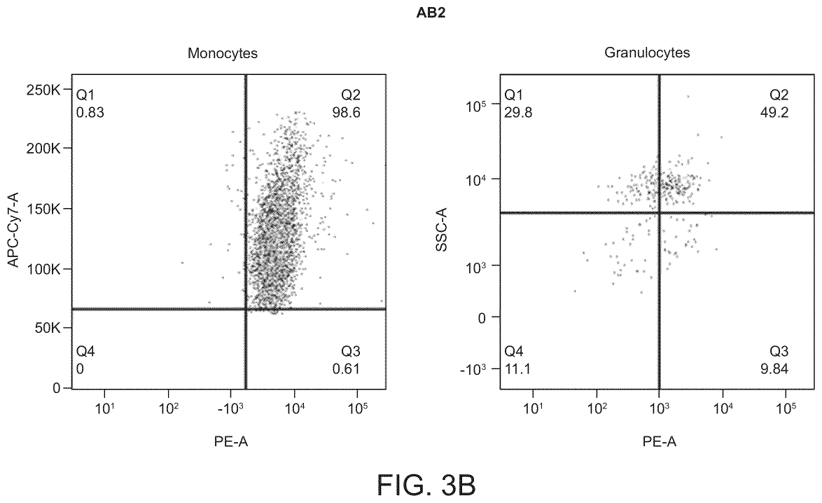

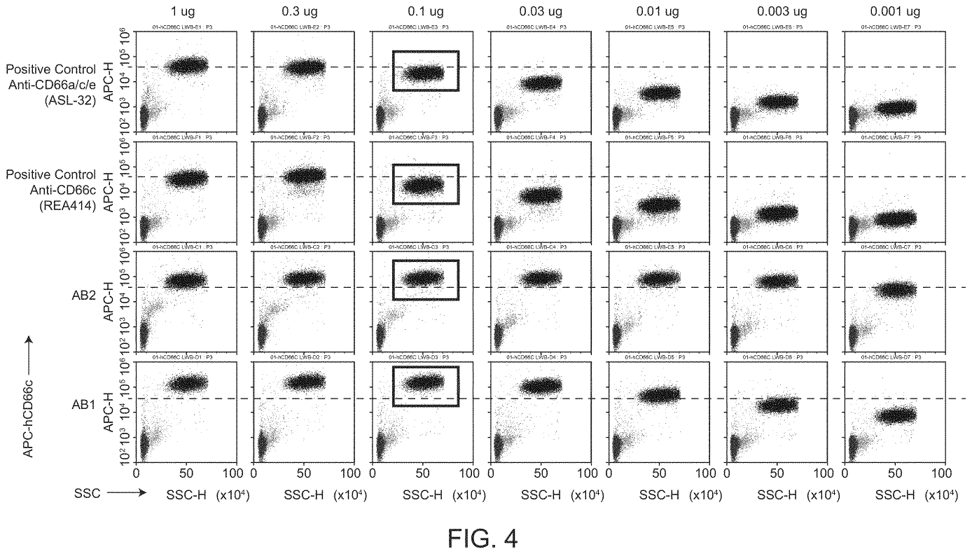

Next, the ability of exemplary antibodies to bind CEACAM6 on white blood cells (lymphocytes, monocytes and granulocytes) isolated from healthy volunteer donors was assed. White blood cells were incubated with anti-CEACAM6 antibody AB1 and AB2, respectively, in whole blood followed by red blood cell lysis. Lysed blood was washed twice with FACS wash buffer and anti-CEACAM6 antibody were detected with either a PE-labeled anti-rat IgG secondary antibody (PE-A in FIGS. 2 and 3) or with an APC-labeled anti-rat IgG secondary antibody (FIG. 4) for 15 minutes. Cells were washed with FACS buffer and analyzed on a BD LSRII flow cytometer. As shown in FIG. 2, exemplary tested anti-CEACAM6 antibodies AB1 (FIG. 2A) and AB2 (FIG. 2B) did not stain lymphocytes based on the very low or no signal density in the quadrants Q2 and Q6. As shown in FIG. 3, both antibodies AB1 (FIG. 3A) and AB2 (FIG. 3B) detected CEACAM6 expression on monocytes and granulocytes based on the high signal density in quadrant Q2. As shown in FIG. 4, on granulocytes both antibodies AB1 and AB2 show significantly brighter signal intensity at 0.1 ug compared to the two reference antibodies REA414 and ASL-32.

### Example 4. Assessment of Antibody Blocking Ability of Exemplary Anti-CEACAM6 Antibodies

This Example describes the ability of exemplary anti-CEACAM6 antibodies to block binding of commercially available anti-CEACAM6 antibodies.

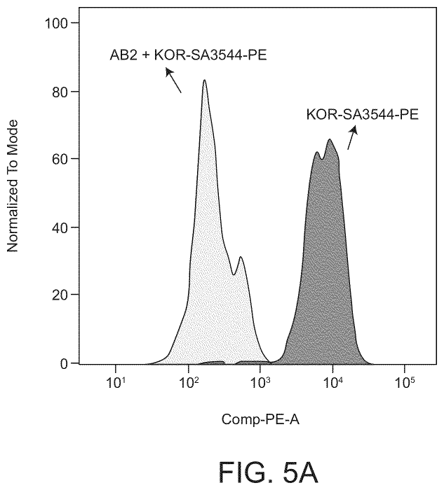

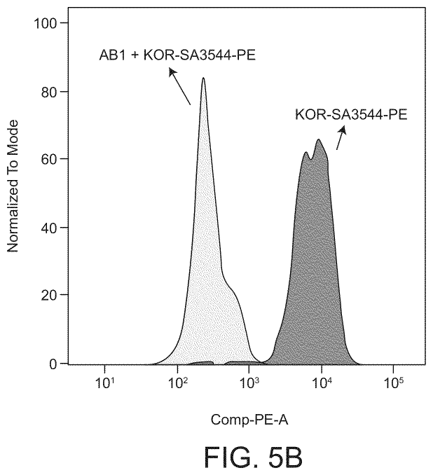

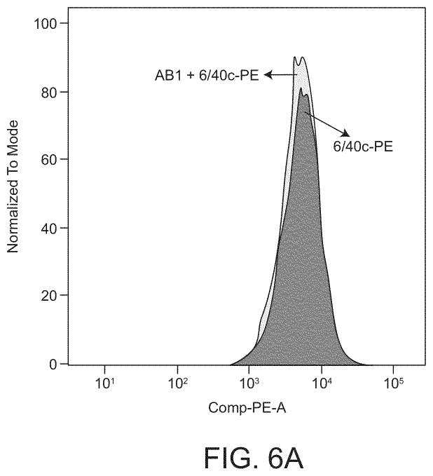

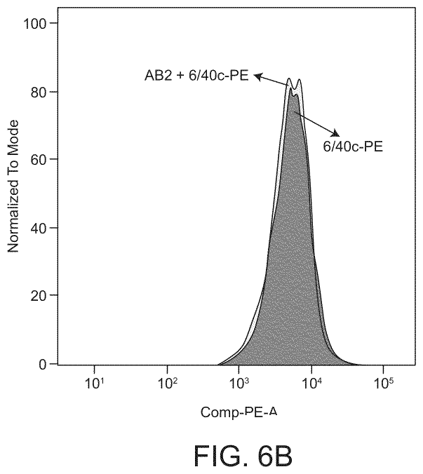

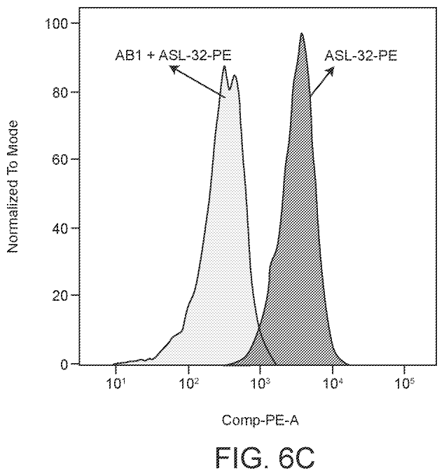

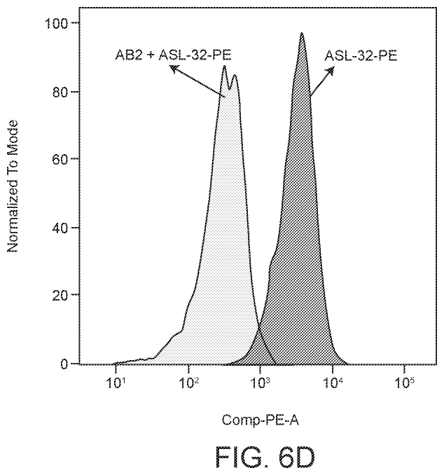

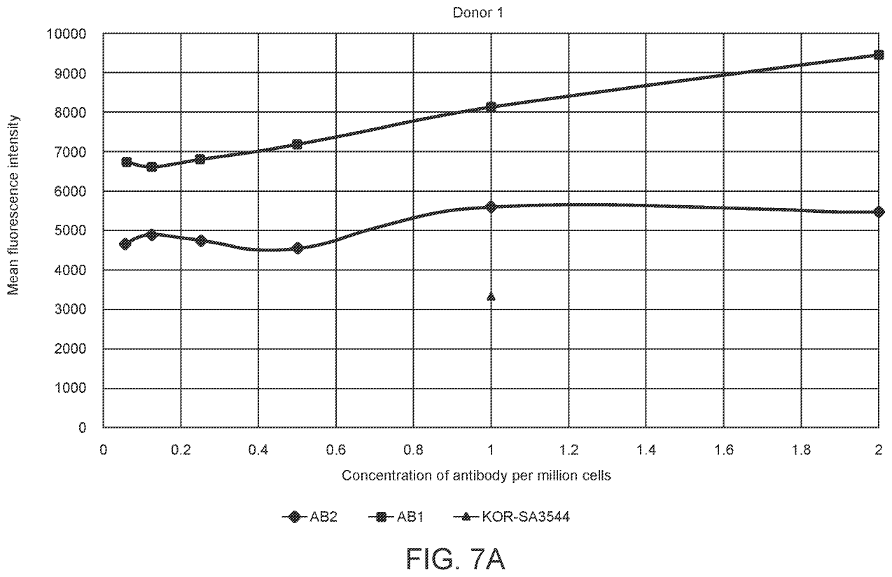

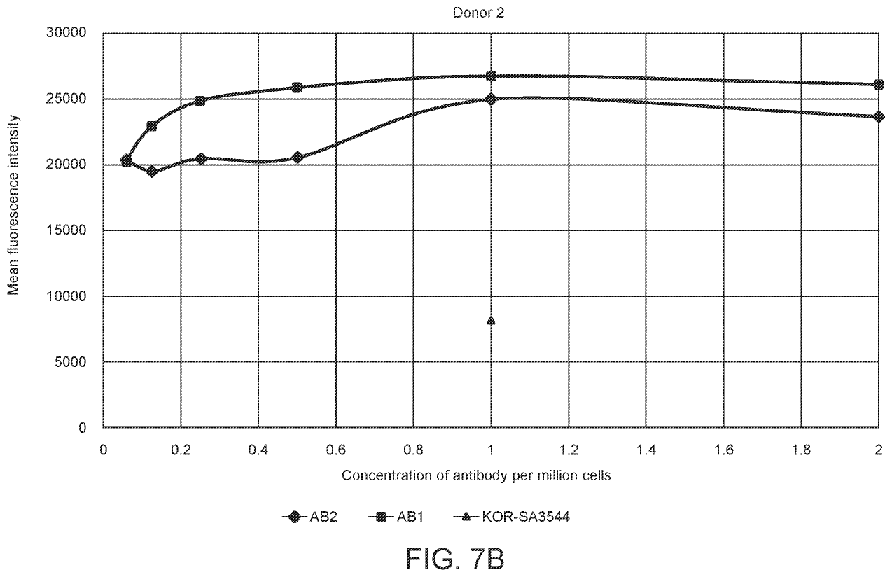

White blood cells (lymphocytes, monocytes and granulocytes) isolated from healthy volunteer donors. White blood cells were incubated with anti-CEACAM6 antibodies for 15 minutes, followed by addition of PE-labeled reference antibody KOR-SA3544. After red blood cell lysis, cells were washed twice with FACS buffer and analyzed on a BD LSRII flow cytometer. As shown in FIG. 5, both antibodies AB1 (FIG. 5B) and AB2 (FIG. 5A) were able to block the binding of reference antibody KOR-SA3544. In a similar experiment, white blood cells were incubated with anti-CEACAM6 antibodies AB1 and AB2 for 15 minutes, followed by addition of PE-labeled reference antibody ASL-32, which binds to CD66a/c/e, or 6/40c. As shown in FIG. 6, both antibodies block binding of the reference antibody ASL-32 to CEACAM6 (FIG. 6C-D) but do not block binding of reference antibody 6/40c to CEACAM8 (FIG. 6A-B). FIG. 7 shows mean fluorescence intensities (y-axis) of the PE-labeled anti-CEACAM6 antibodies AB1 and AB2 at different concentrations (x-axis: concentration of antibody per million cells), compared to the mean fluorescence intensity of the PE-labeled reference antibody KOR-SA3544 (measured at a single concentration). Both anti-CEACAM6 antibodies AB1 and AB2 show a higher mean fluorescence intensity than KOR-SA3544 at a concentration of antibody per million cells of 1.

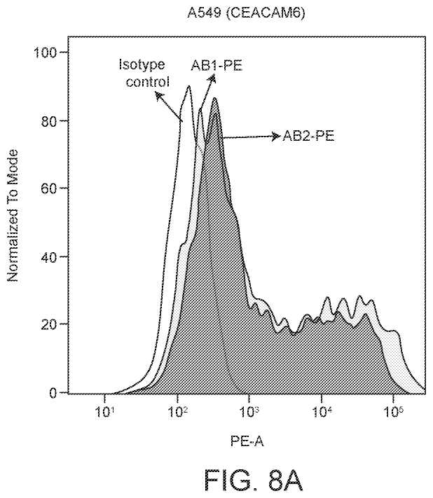

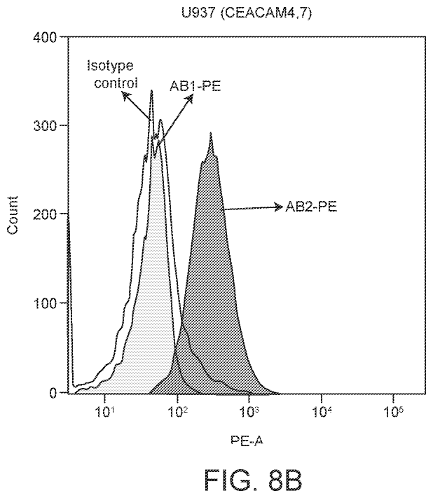

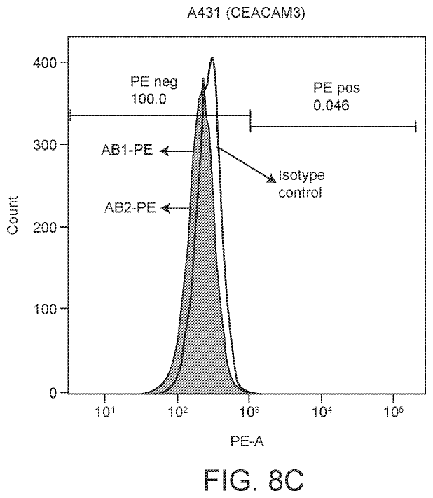

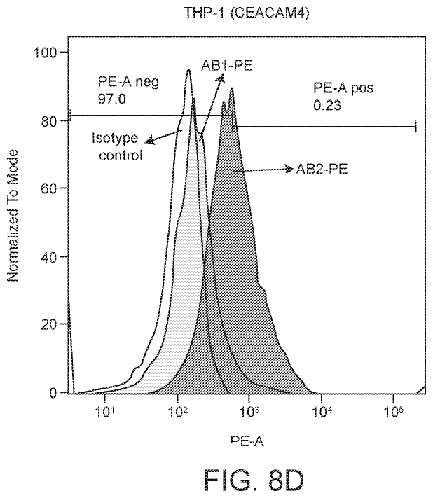

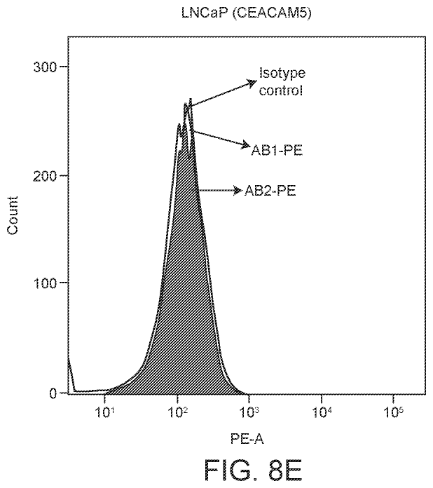

To confirm the specificity of the two anti-CEACAM6 antibody to CEACAM6 and assess cross-reactivity with other CEACAM family members, anti-CEACAM6 antibodies AB1 and AB2 were tested on the CEACAM6-expressing human lung carcinoma cell line A549 (ATCC A549 CCL-185), as well as the CEACAM4 and 7-expressing human monocytic cell line U-937 (ATCC U-937 CRL-1593.2), the CEACAM3-expressing epidermoid carcinoma cell line A431 (ATCC CRL-1555), the CEACAM4-expressing human monocytic leukemia cell line THP-1 and the CEACAM5-expressing human prostate carcinoma cell line LNCaP (ATCC CRL-1740). As shown in FIG. 8, anti-CEACAM6 antibody AB1 specifically reacted with CEACAM6 based on the right-shift of the peak compared to the isotype control in FIG. 8A and did not cross-react with any other CEACAM family members based on the absence of any right-shift of the peak compared to the isotype control in FIG. 8B-D. Antibody AB2 specifically reacted with both CEACAM6 and CEACAM4 based on the right-shift of the peak compared to the isotype control in FIGS. 8A, B and D.

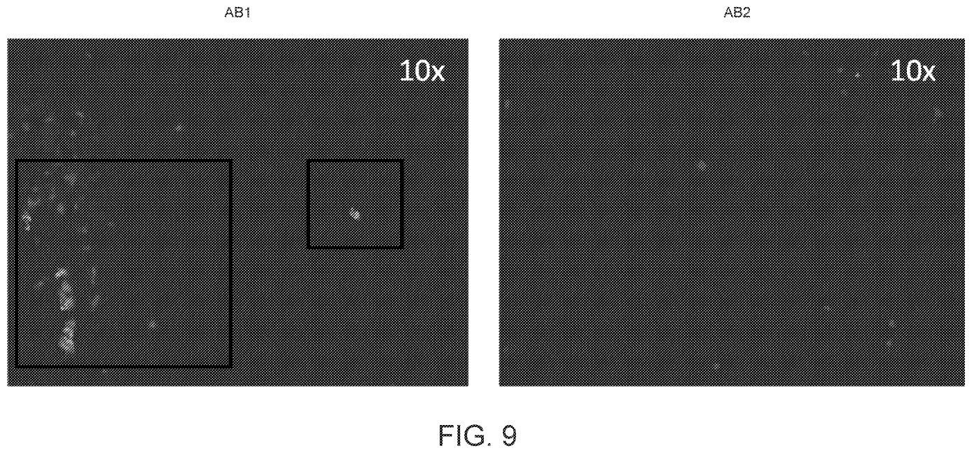

In another experiment, the ability of exemplary anti-CEACAM6 antibodies AB1 and AB2 to stain CEACAM6 protein in formalin-fixed paraffin-embedded (FFPE) samples was assessed by immunohistochemistry (IHC). 5 μm sections of FFPE samples of human colon were deparaffinized using xylene, and rehydrated in graded ethanol. Heat mediated antigen retrieval was performed using Sodium Citrate pH 6.0 at 90 °C for 30 minutes. Samples were permeabilized with 0.1% Triton X-100 in PBS for 30 minutes and blocked with 5% FBS in PBS for 1 h. Samples were stained with 5 g/ml of purified exemplary anti-human CEACAM6 antibodies AB1 or AB2 over night at 4 °C. Tissue sections were then washed with PBS and stained with 2.5 ug/ml of Alexa-555 conjugated anti-rat IgG dye for 1 hour at room temperature in the dark, followed by two washes in PBS. Samples were mounted using Antifade gold with DAPI and imaged using with Metamorph software and analyzed using image J. As shown in FIG. 9, anti-CEACAM6 antibody AB1 is capable of staining CEACAM6 protein in human colon paraffin sections based on the detected fluorescent signals in the left fluorescence image in FIG. 9, while anti-CEACAM6 antibody AB2 is not capable of staining CEACAM6 in human colon paraffin sections (right fluorescence image).

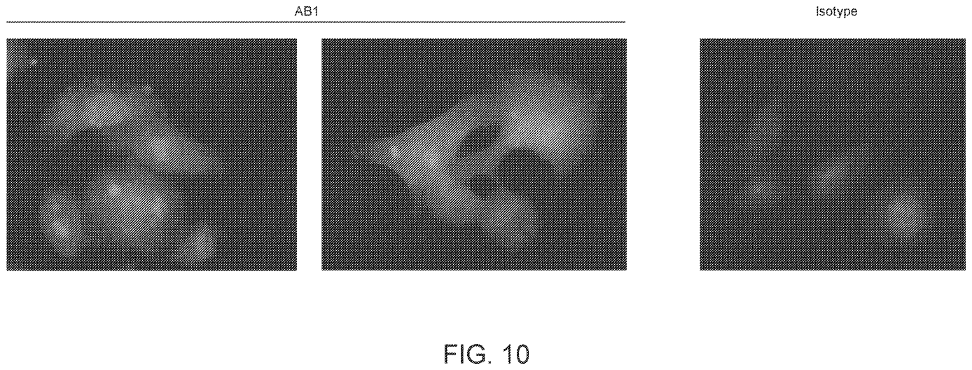

In another experiment, the ability of exemplary anti-CEACAM6 antibody AB1 to stain CEACAM6 in fixed cells was assessed by immunohistochemistry (ICC). The human lung adenocarcinoma cell line A549 was grown on 96-well plates with coverslip bottom and fixed with Fixation Buffer for 30 minutes. Cells were washed twice with PBS and stained with antibody AB1 followed by Alexa Fluor®555 anti-rat IgG antibody. Nuclei were counter-stained with DAPI dye. Cells were imaged using a 40× objective. As shown in FIG. 10, anti-CEACAM6 antibody AB1 detected CEACAM6 protein expression in A549 cells based on the fluorescent signals in the left and middle fluorescence image in FIG. 10 compared to no fluorescent signals in the isotype control (FIG. 10, right).

### Example 5. Assessment of Functional Activity of Exemplary Anti-CEACAM6 Antibodies

This Example describes the functional assessment of exemplary anti-CEACAM6 antibody AB1 as measured by the inhibition of CEACAM6 activity.

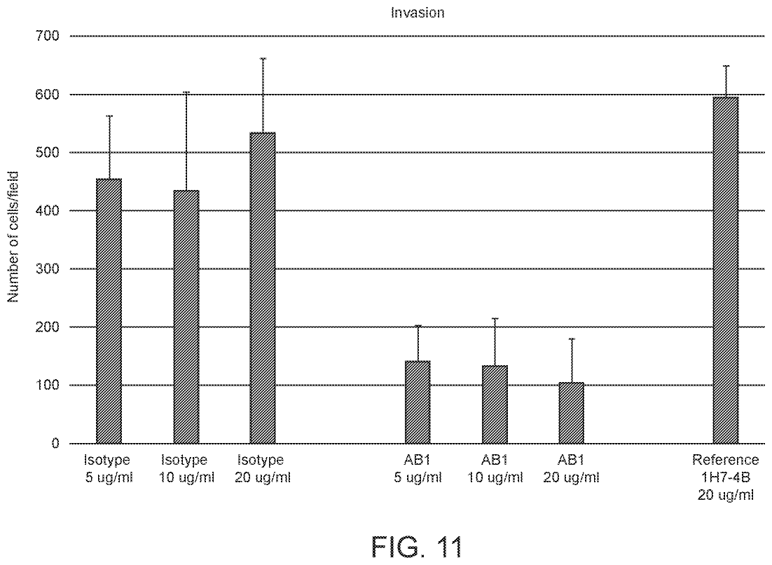

This Example describes the functional assessment of exemplary anti-CEACAM6 antibody AB1 as measured by its effect on cancer cell invasion, as compared to a commercially available antibody (Reference 1H7-4B). The effect of anti-CEACAM6 on the ability of blocking cells to invade through the extracellular matrix was tested by a Matrigel invasion assay. The human lung carcinoma cell line A549 (ATCC A549 CCL-185) was serum starved for 24h, after which 5×105 cells were resuspended in serum-free DMEM media with AB1 at or the isotype control antibody (Isotype) at 20, 10 and 5 μg/ml seeded onto the top well of Corning BioCoat Matrigel Invasion Chambers. DMEM media with 20% serum was placed in the bottom well as chemoattractant. After 16h, cells were removed from the top well with a cotton swab and cells that had migrated to the bottom of the membrane were counted. Each experiment was performed in duplicates and 3 images were counted using 910xo objective for each condition using the cell counter feature in Image J software. As shown in FIG. 11, AB1 blocked A549 cells to invade the Matrigel and to migrate to the bottom of the membrane at 20, 10 and 5 ug/ml based on a significantly reduced number of cells per field counted for all three concentrations. In contrast, the commercially available antibody 1H7-4B did not block invasion at 20 ug/ml (Reference 20 μg/ml) based on the comparably high number of cells per field counted in comparison to the isotype control (Isotype 20 μg/ml).

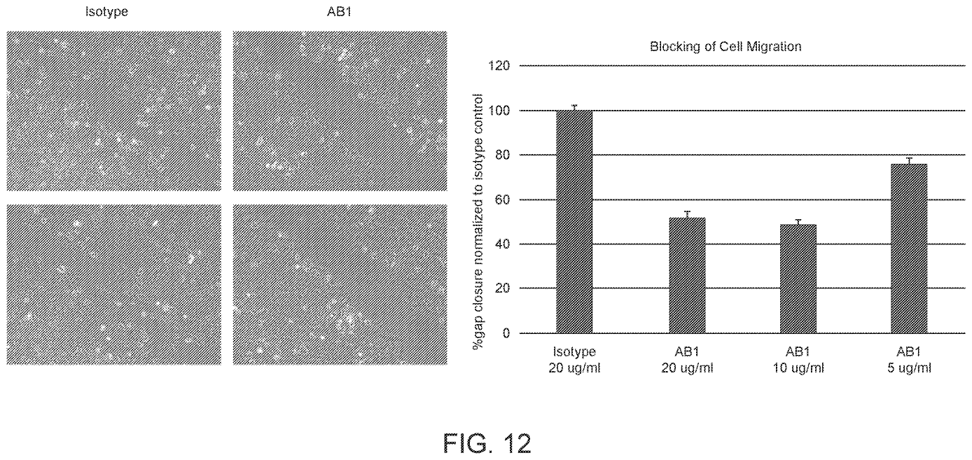

The ability of anti-CEACAM6 antibody AB1 to inhibit cancer cell migration was tested by a wound healing assay. The human lung adenocarcinoma cell line A549 was grown to confluency, and a line was scratched using a 10 ul pipette tip. Anti-CEACAM6 antibody AB1 was added at 20, 10 or 5 ug/ml. Rat IgG2a, k isotype control antibody was used as control. After 16 h, the width of the gap was measured using Image J, and % gap closure was calculated normalized to the isotype control. As shown in FIG. 12, anti-CEACAM6 antibody AB1 inhibited lung cancer cell migration by more than 50% at 20 and 10 ug/ml.

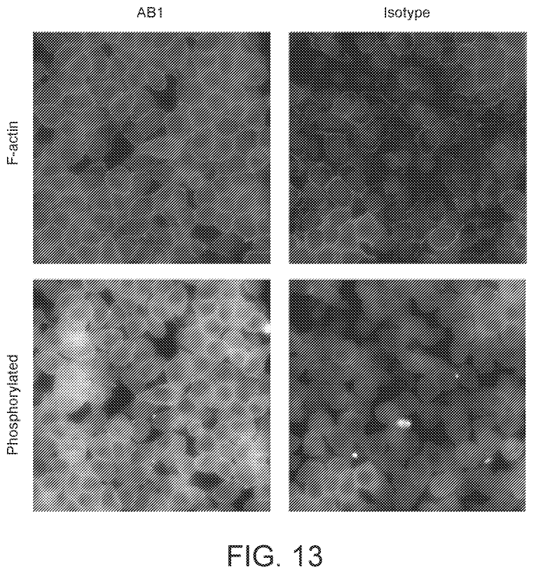

In another experiment, the ability of anti-CEACAM6 antibody AB1 to induce intracellular signaling through CEACAM6 activation was tested. The effect of antibody AB1 to induce intracellular signaling by cross-linking was tested on the human lung adenocarcinoma cell line A549. Cells were grown on 96-well plates with coverslip bottom and treated with 5 μg/ml anti-human CEACAM6 antibody AB1 or rat IgG2a, k isotype control antibody for 15 minutes at 37 °C followed by anti-rat IgG secondary antibody (5 ug/ml) for 30 minutes at 37 °C. Cells were fixed with Fixation Buffer for 30 minutes. Cells were washed twice with 1× Intracellular Staining Permeabilization Wash Buffer and then stained with anti-AKT Phospho (Ser473) antibody from mouse followed by Alexa Fluor®555 anti-mouse IgG dye, Flash Phalloidin™ Red 594 dye and DAPT dye. Flash Phalloidin™ Red 594 dye is a probe for imaging and stabilizing filamentous F-actin in fixed and permeabilized cells, thereby providing structural and volumetric context to the cell. As shown in FIG. 13, cross-linking of CEACAM6 with the anti-CEACAM6 antibody AB1 induced intracellular signaling which resulted in increased phosphorylation of the Akt kinase and increased the actin polymerization. This conclusion is based on the increase in fluorescence signals and intensity around the nuclei when comparing the AB1 fluorescence images (FIG. 13 left top and bottom) with the corresponding isotype control fluorescence images (FIG. 13 right top and bottom).

The examples herein are provided to illustrate embodiments of the disclosure but not to limit its scope. Other variants of the disclosure will be readily apparent to one of ordinary skill in the art and are encompassed by the appended claims. All publications, databases, internet sources, patents, patent applications, and accession numbers cited herein are hereby incorporated by reference in their entireties for all purposes.

What is claimed is:

1. An isolated antibody or antigen binding fragment thereof that binds CEACAM6 or a portion thereof, wherein the antibody comprises: (i) an immunoglobulin heavy chain comprising a set of heavy chain complementarity determining region (CDR) amino acid sequences, CDRH1, CDRH2, and CDRH3; and, (ii) an immunoglobulin light chain comprising a set of light chain CDR amino acid sequences, CDRL1, CDRL2, and CDRL3, wherein the sets of heavy chain and light chain CDRs are each chosen from the same of set 1 or 2:

| | CDRH1 | CDRH2 | CDRH3 | CDRL1 | CDRL2 | CDRL3 |
|---|---|---|---|---|---|---|
| CDR | (SEQ ID | (SEQ ID | (SEQ ID | (SEQ ID | (SEQ ID | (SEQ ID |
| Set | NO:) | NO:) | NO:) | NO:) | NO:) | NO:) |
| 1 | GFSLSTY | IWWDDD | ARILLGF | KSISNT | SGS (16) | QHYNEY |
| | GMG (12) | K (13) | DY (14) | (15) | | PLT (17) |
| 2 | GFTFSNY | IANSGGT | TTLKFGA | QSLLYNE | WAS (10) | QQYYIFP |
| | G (6) | T (7) | GGFAY | NKKNY | | NT (11) |
| | | | (8) | (9) | | |

2. The antibody or antigen binding fragment thereof of claim 1, wherein the immunoglobulin heavy chain comprises the amino acid sequence set forth in SEQ ID NO: 4, or a sequence having at least 80% amino acid sequence identity to SEQ ID NO: 4, and wherein the immunoglobulin light chain comprises the amino acid sequence set forth in SEQ ID NO: 5, or a sequence having at least 80% amino acid sequence identity to SEQ ID NO: 5.

3. The antibody or antigen binding fragment thereof of claim 1, wherein the immunoglobulin heavy chain comprises the amino acid sequence set forth in SEQ ID NO: 2, or a sequence having at least 80% amino acid sequence identity to SEQ ID NO: 2, and wherein the immunoglobulin light chain comprises the amino acid sequence set forth in SEQ ID NO: 3, or a sequence having at least 80% amino acid sequence identity to SEQ ID NO: 3.

4. A diagnostic antibody or antigen binding fragment thereof comprising the antibody or antigen binding fragment thereof of any of claims 1-3.

5. A kit comprising the antibody or antigen binding fragment thereof of any one of claims 1-4.

6. A pharmaceutical composition comprising the antibody or antigen binding fragment thereof of any of claims 1-4, and a pharmaceutically acceptable excipient.

7. An isolated nucleic acid comprising a nucleotide sequence that encodes the immunoglobulin heavy chain variable domain of the antibody or antigen binding fragment thereof of any of claims 1-4.

8. An isolated nucleic acid comprising a nucleotide sequence that encodes the immunoglobulin heavy chain variable domain of the antibody or antigen binding fragment thereof of any of claims 1-4.

9. A recombinant expression vector comprising the isolated nucleic acid of claim 7 and/or the isolated nucleic acid of claim 8.

10. A host cell comprising the nucleic acid of claim 7 and/or the nucleic acid of claim 8, or the expression vector of claim 8.

11. An isolated nucleic acid comprising a nucleotide sequence, wherein the nucleotide sequence encodes an immunoglobulin heavy chain comprising SEQ ID NO: 4 or a sequence having at least 90% sequence identity to the sequence set forth in SEQ ID NO: 4, or an immunoglobulin light chain comprising SEQ ID NO: 5 or a sequence having at least 90% sequence identity to the sequence set forth in SEQ ID NO: 5.

12. A recombinant expression vector comprising the isolated nucleic acid of claim 11.

13. A host cell comprising the nucleic acid of claim 11 or the expression vector of claim 12.

14. An isolated nucleic acid comprising a nucleotide sequence, wherein the nucleotide sequence encodes an immunoglobulin heavy chain comprising SEQ ID NO: 2 or a sequence having at least 90% sequence identity to the sequence set forth in SEQ ID NO: 2, or an immunoglobulin light chain comprising SEQ ID NO: 3 or a sequence having at least 90% sequence identity to the sequence set forth in SEQ ID NO: 3.

15. A recombinant expression vector comprising the isolated nucleic acid of claim 14.

16. A host cell comprising the nucleic acid of claim 14 or the expression vector of claim 15.

17. A recombinant expression vector comprising a first expression cassette and a second expression cassette, wherein the first expression cassette comprises a nucleic acid molecule comprising a nucleotide sequence that encodes the immunoglobulin heavy chain variable domain of any one of claims 1-4, and the second expression cassette comprises a nucleic acid molecule comprising a nucleotide sequence that encodes an immunoglobulin light chain variable domain of the antibody or antigen binding fragment thereof of any one of claims 1-4.

18. A host cell comprising the recombinant expression vector of claim 17.

19. A method of detecting CEACAM6, the method comprising: contacting a sample with the antibody or antigen binding fragment thereof of any of claims 1-4, under conditions to bind said antibody or antigen binding fragment thereof to a CEACAM6 receptor in said sample, wherein the binding generates the production of a receptor/antibody or antigen binding fragment thereof complex.
